(19) United States
(12) Patent Application Publication
(10) Pub. No.: US 2009/0263418 A1
(43) Pub. Date: Oct. 22, 2009

(54) MEANS AND METHODS FOR MANIPULATING SEQUENTIAL PHAGOLYSOMALCYTOSOLIC TRANSLOCATION OF MYCOBACTERIA, AND USES THEREOF

(76) Inventors: Nicole Neeltje Speelman-Van Der Wel, Utrecht (NL); Michael Barry Brenner, Newton, MA (US); Jacobus Peter Johannes Peters, Amsterdam (NL)

Correspondence Address: RISSMAN HENDRICKS & OLIVERIO, LLP, 100 Cambridge Street, Suite 2101, BOSTON, MA, 02114, US

(21) Appl. No.: 12/307,948

(22) PCT Filed: Jun. 29, 2007
(86) PCT No.: PCT/NL07/50323

§ 371 Date: Apr. 9, 2009

(30) Foreign Application Priority Data
Jul. 10, 2006 (NL) PCT/NL2006/000349

(51) Int. Cl. A61K 39/04; C12Q 1/68; C12Q 1/20; C12N 15/87; C12N 1/21

(52) U.S. Cl. 424/200.1; 435/6; 435/253.1; 435/471; 435/252.3

(57) ABSTRACT

Mycobacteria such as M. tuberculosis and M. leprae are considered to be prototypical intracellular bacilli that have evolved strategies to enable growth in the intracellular phagosomes of the host cell. By contrast, we show that lysosomes rapidly fuse with the virulent M. tuberculosis and M. leprae— containing phagosomes of human monocyte-derived dendritic cells and macrophages. After 2 days, M. tuberculosis progressively translocate from phagolysosomes into the cytosol where they replicate. Cytosolic entry is also observed for M. leprae but not for the vaccine strain, M. bovis BCG, or killed mycobacteria, and is dependent upon secretion of the mycobacterial gene products CFP-IO and ESAT-6 of the RDI region. The present invention further provides means and methods for using these findings in therapeutic and immunogenic compositions.

The invention relates to the medical and veterinarian field. More, in particular the invention relates to pathogenesis of mycobacteria and the use of mycobacterial strains as a starting material for vaccines.

Successful bacterial pathogens access and establish in vivo niches that are suitable to bacterial replication. For commensals, competition for nutrients determines outcome; while for pathogens bacterial survival occurs in the face of innate and adaptive immune responses targeted at their elimination. Selective pressures imparted by interactions with hosts have contributed to pathogen evolution through the acquisition of genes that enable immune evasion and allow bacterial survival and replication. In many cases, this has occurred through the acquisition of large blocks of genes encoded on mobile genetic elements that can be readily transmitted between bacterial strains. Such elements include genes encoding bacterial exotoxins, such as Cholera toxin and Diptheria toxin, and genes encoding Type III and Type IV secretion systems that secrete effector proteins into host cells and modulate host cell functions.

Initial host-pathogen encounters include bacterial interactions with epithelial and mucosal tissues that serve as physical barriers to invasion and infection. Additionally, host phagocytes, such as macrophages and dendritic cells (DCs) have a significant role in innate host resistance to infection and contribute to the generation of adaptive immune responses. These myeloid cells internalize microbes into membrane bound organelles termed phagosomes that mature and fuse with lysosomes. Phagolysosome fusion creates an acidic environment rich in hydrolytic enzymes that degrade and kill bacteria. Moreover, proteolysis of bacterial proteins in these compartments generates antigenic peptides that may elicit MHC Class II restricted T cell responses. Thus, bacterial evasion strategies targeted at blocking phagolysosome fusion may result in both enhanced survival and delay in the initiation of adaptive immunity.

Intracellular pathogens commonly avoid lysosomal fusion through the manipulation of host signal transduction pathways and alteration of endocytic traffic resulting in privileged replicative niches. Salmonella species impede the acquisition of lysosomal hydrolases and reactive oxygen intermediates through the actions of Type III secretion system effector proteins, and reside in an acidified endosome suitable for growth (Waterman and Holden, 2003). Legionella pneumophila induces phagosomes to fuse with secretory vesicles from the ER and Golgi and create an early secretory compartment that is devoid of degradative enzymes and rich in nutrients (Roy and Tilney, 2002; Zamboni et al., 2006). In contrast, Listeria monocytogenes and Shigella flexneri lyse the phagosomal membrane and escape from the endocytic system into the host cytosol where they replicate and are able to spread to neighbouring cells via actin-based motility (Stevens et al., 2006). In these cases, pathogens escape hydrolytic enzymes and the MHC Class II antigen presentation pathway, yet by entering the cytosol, their products may be detected by the MHC Class I antigen-processing pathway. Nearly all intracellular pathogens have specialized to manage their fates as “endosomal” or “cytosolic” pathogens.

It is currently thought that one of the most successful human bacterial pathogens, Mycobacterium tuberculosis, persists and replicates within the phagosomes of macrophages where it prevents lysosomal fusion and maintains extensive communication with early endosomal traffic in a fashion that is thought to provide access to nutrients for survival and growth (Russell et al., 2002; Vergne et al., 2004). In the present work, we determined the localization of M. tuberculosis and M. leprae in human myeloid DCs and macrophages in order to better understand the natural history of intracellular infection of these organisms.

Using cryo-immunogold electron microscopy we find that at early time points after phagocytosis, M. tuberculosis phagosomes fuse with late endocytic multivesicular bodies and lysosomes and at steady-state the bacteria reside in a phagolysosomal compartment. This localization correlates with static bacterial growth over the same time period. Surprisingly, at later time points M. tuberculosis translocation from phagolysosomes into the host cytosol at which time bacterial titers in infected cultures begin to increase. A similar phenotype was also detected for M. leprae. Phagolysosomal egression requires live bacteria and does not occur following infection with BCG. M. tuberculosis mutants defective for the synthesis or secretion of the CFP10 and ESAT6 proteins remain restricted to the phagolysosome indicating a role for the specialized secretion system Esx-1 which is partly encoded in the genomic region of difference RD1. Thus, translocation into the cytosol appears to provide M. tuberculosis a replicative niche separated from degradative lysosomes and the MHC Class II presentation pathway.

In one aspect the invention provides a method for determining whether a product of a gene of a mycobacterium is involved in translocation of said mycobacterium from the phagosome to the cytosol of a host cell, said method comprising altering said gene product and/or expression of said gene product in said mycobacterium and determining whether said translocation of said mycobacterium in said host cell is affected. Equivalent to altering said gene product and/or expression of said gene product in said mycobacterium is of course to select an already existing mutant mycobacterium wherein said gene product and/or expression of said gene product is altered with respect to the model mycobacterium, preferably the wild type. In this way it is possible to identify genes and gene products that are involved in the translocation to the cytosol. The selected genes or gene products can be promoting the translocation or play a part in inhibiting the translocation. For instance, it has been observed that translocation is a timed process in that it is observed only a few days after infection of the host cell. It has been found that genes and gene products of the specialized secretion system Esx-1 are involved in promoting the translocation. Thus genes and gene products that counteract this secretion system, or the secretion of one or more of the relevant gene products encoded by it, have a repressive effect on translocation and thus promote maintenance of the phagosomal state. Host cells infected with a mycobacterium expressing CFP10, ESAT6 and/or EspA exhibit a higher level of apoptosis than comparable host cells infected with a mycobacterium that does not express CFP10, ESAT6 and/or EspA. Using a method of the invention it is possible to identify both genes and gene products that promote the translocation and genes and gene products that inhibit said translocation.

In a preferred embodiment, said gene is a gene from a region of difference (RD) between mycobacterium tuberculosis and Bacille Calmette Guerin (BCG), or from a corresponding region in another mycobacterium species. In the present invention it has been observed that BCG is a strain of mycobacterium that is deficient in translocation. It survives and replicates predominantly in the phagosomes of infected cells. BCG is a strain that has been cultured extensively in vitro, and likely as a result of this has lost selected parts of its genome, when compared to wild type species such as mycobacterium bovis and tuberculosis. These selected parts of the genome have been characterised and termed ‘regions of difference’. Thus far, 14 of such regions of difference have been characterized. In the present invention these regions of difference have been scrutinized for the presence of genes and their encoded products that affect, and preferably, promote the translocation into the cytosol. In a preferred embodiment therefore, a product of gene for which it is determined whether it is involved in translocation of said mycobacterium from the phagosome to the cytosol of a host cell, comprises a product of a gene from a region of difference (RD) between mycobacterium tuberculosis and a Bacille Calmette Guerin (BCG) strain. It is known that mycobacterial species share a great deal of homology with each other. BCG, for instance, as mentioned above, is a strain derived from mycobacterium bovis. BCG has effectively been used to immunize humans and particularly juveniles against mycobacterium tuberculosis infection. This is only possible when mycobacterium bovis, and mycobacterium tuberculosis share a large part of their immunogenic epitopes. For the present invention it is thus also possible to select a homologues gene in a non-bovis strain, based on the difference between BCG and bovis. In other words, to find the corresponding gene that is in a region of difference in mycobacterium tuberculosis in another species of mycobacteria. This corresponding gene encodes a gene product that shares at least 90% sequence identity with the RD gene in mycobacterium tuberculosis. Thus in a method of the invention for determining whether a gene or a gene product is involved in translocation to the cytosol, said gene is preferably a gene from a region of difference (RD) between mycobacterium tuberculosis and Bacille Calmette Guerin (BCG), or from a corresponding region in another mycobacterium species, or a gene from a corresponding region in another mycobacterium species. In a preferred embodiment, said other (corresponding) mycobacterium species is selected from mycobacterium bovis, kansasii, africanum, leprae, smegmatis or marinum.

Gene products involved in promoting translocation are preferably selected from RD1, from the specialized secretion system Esx-1 and preferably selected from CFP10, ESAT6 or EspA. Said gene product is preferably a mycobacterium bovis, mycobacterium tuberculosis, kansasii, africanum, leprae, smegmatis or marinum gene product. The gene product can also be a chimeric protein having an amino sequence that is derived from CFP10, ESAT6 or EspA from two or more mycobacterial strains, or species. Such a consensus CFP10, ESAT6 or EspA is also part of the invention. Thus the invention further provides a method for reducing the phago-cytosolic translocation of a mycobacterium comprising at least reducing the expression of (consensus) CFP10, ESAT6 or EspA in said mycobacterium. The expression can be reduced by altering the promoter strength, or it can be reduced by mutating said gene such that the functionality of the gene product is reduced or absent in the thus manipulated mycobacterium. Preferably the expression is reduced by deleting the gene encoding CFP10, ESAT6 or EspA either in whole or in part from the genome. Said part is defined such that the translocation is inhibited. However, other alterations are within the skill of the person skilled in the art. For instance, frame shift mutations due to insertions are also possible.

The invention further provides a method for enhancing phago-cytosolic translocation of a CFP10, ESAT6 and/or EspA deficient mycobacterium, said method comprising providing said mycobacterium with CFP10, ESAT6 and/or EspA. A mycobacterium is deficient in CFP10, ESAT6 and/or EspA when the expression of said product in said mycobacterium is either lacking or suboptimal. It is of course only necessary to provide the gene product that is missing or which presence is suboptimal in said mycobacterium. When CFP10, ESAT6 and EspA a preferred embodiment said bacterium is provided with CFP10, ESAT6 and/or EspA. Preferably, said CFP10, ESAT6 and/or EspA is from the same mycobacterium species as to which it is provided. However, can also be from a different mycobacterium species, or be a consensus CFP10, ESAT6 and/or EspA. Equivalent to CFP10, ESAT6 and/or EspA is a protein that shares at least 90% sequence identity with CFP10, ESAT6 and/or EspA of a mycobacterial species and that shares the same translocation promoting function in kind, not necessarily in amount. Said mycobacterium species is preferably a mycobacterium bovis, mycobacterium tuberculosis, kansasii, africanum, leprae, smegmatis or marinum. Said mycobacterium may also be a strain derived from one of these species, preferably a BCG strain. The invention thus further provides a method for generating a recombinant BCG strain comprising providing BCG or a derivative thereof with CFP10, ESAT6 and/or EspA. Also provided is a BCG strain comprising CFP10, ESAT6 and/or EspA. Such a BCG strain is particularly suited for the preparation of an immunogenic composition as MHC-I type immunogenicity is enhanced when compared to the original BCG strain, prior to providing it with CFP10, ESAT6 and/or EspA. CFP10, ESAT6 and/or EspA can be provided to said mycobacterium in a number of ways. It is preferably provided by providing said mycobacterium through insertion therein of a nucleic acid encoding CFP10, ESAT6 and/or EspA. The nucleic acid may be a plasmid or other extrachromosomal nucleic acid. In addition the nucleic acid may be integrated into the chromosomal DNA of said mycobacterium. Said nucleic acid is preferably inserted into said mycobacterium together with the necessary signals for allowing expression of CFP10, ESAT6 and/or EspA. However, using recombinant DNA technology it is also possible to insert a coding region in an already present expression cassette. In a preferred embodiment said nucleic acid encoding CFP10, ESAT6 and/or EspA is provided to said mycobacterium in the absence of at least one other protein coding region of RD1. The invention further provides a recombinant BCG mycobacterium comprising a nucleic acid encoding CFP10, ESAT6 and/or EspA, a consensus CFP10, ESAT6 and/or EspA or an equivalent thereof that shares at least 90% sequence identity with CFP10, ESAT6 and/or EspA of a mycobacterial species and that shares the same translocation promoting function in kind, not necessarily in amount. In a preferred embodiment said mycobacterium is provided with an RD1 region, preferably an extended RD1 region.

Further provided is a method for producing a mycobacterium that is substantially deficient in phago-cytosolic translocation comprising functionally reducing the expression of CFP10, ESAT6 and/or EspA in said mycobacterium. Functional reduction of expression is preferably obtained by mutating and/or removing the gene encoding CFP10, ESAT6 and/or EspA such that substantially no functional CFP10, ESAT6 and/or EspA is produced by said mycobacterium.

Further provided is an attenuated mycobacterium comprising a nucleic acid encoding CFP10, ESAT6 and/or EspA further comprising a heterologous nucleic acid for inhibiting cytosolic replication and/or cytosolic translocation of said mycobacterium. Mycobacteria lacking CFP10, ESAT6 and/or EspA lack the capacity to facilitate translocation to the cytosol and thereby exhibit at least reduced translocation and replication in a host cell. This phenotype can be obtained by reducing the expression of CFP10, ESAT6 and/or EspA in said mycobacterium. Several methods are available to the person skilled in the art. In a preferred embodiment a heterologous nucleic acid is inserted into said mycobacterium to reduce said expression. Provided is therefore an attenuated mycobacterium comprising a nucleic acid encoding CFP10, ESAT6 and/or EspA further comprising a heterologous nucleic acid for inhibiting cysolic replication and/or cytosolic translocation of said mycobacterium in a eukaryotic host cell. Said heterologous nucleic acid is preferably present in the genome or a plasmid. Said heterologous nucleic acid is preferably a promoter, preferably a regulatable promoter. Said heterologous nucleic acid is preferably a nucleic acid from another species, preferably not from a mycobacterium species. It is preferred that the expression of said CFP10, ESAT6 and/or EspA is under transcriptional control of said regulatable promoter. In another preferred embodiment a gene for replication of said mycobacterium is under control of said regulatable promoter. A preferred example of a replication of the invention is dnaA (Greendyke et al (2002) Vol 148: pp 3887-3900). In a preferred embodiment said regulatable promoter is regulatable through the administration of a compound. A preferred but not limiting example is the tet-operon system. The tetracycline system and analogues acting systems have been developed to regulate the action of the promoter by means of a compound that can be added to the surrounding fluid of the cell (see for instance Gossen and Bujard (2002) Annual Review of Genetics. Vol. 36: 153-173.). In a preferred embodiment said mycobacterium further comprises a nucleic acid encoding the transacting factor that dependent on the presence of said compound binds to and regulates said promoter. In this way cysolic replication and/or cytosolic translocation is dependent on the presence or absence of said compound. This property can be used to generate for instance a mycobacterium that is capable of efficient replication and/or translocation in a host thereby enabling the generation of a robust immune response, whereupon the host can be protected from further consequences of the administration by downregulating the expression through the action of the regulatable promoter. Inhibiting cytosolic replication and/or cytosolic translocation impedes the persistence of the infection and allows the host to more easily clear the body from infected cells. Regulatable systems are available that allow expression from the regulatable promoter in the absence or in the presence of said compound. Thus the invention further provides a method for immunizing an individual with a mycobacterium comprising providing said individual with a mycobacterium comprising a heterologous nucleic acid comprising a regulatable promoter of the invention and downregulating expression from said promoter when expression from said promoter is no longer desired. Preferably when a sufficiently strong immune response has been obtained. This can be achieved by stopping administration of said compound in case of a promoter that is active in the presence of said compound, or by administering said compound in case of a promoter that is active in the absence of said compound. This regulatable system can also be applied to a bacterium of the invention.

In a preferred embodiment said mycobacterium is a mycobacterium tuberculosis, bovis or leprae.

Mycobacteria have been used in the past to produce immunogenic composition either to obtain a strong immune response to the mycobacterium itself or to produce a strong immune response to a co-delivered foreign immunogen. In the latter case, it is often referred to as adjuvant. The present invention thus further provides the use of a mycobacterium of the invention for producing an immunogenic composition. In the latter case, the foreign antigen is supplied as an immunogen, or alternatively, said mycobacterium is provided with a nucleic acid encoding said foreign antigen. In one embodiment said foreign antigen comprises a human protein, preferably a human disease associated protein, preferably a tumour associated protein, such as PRAME, MAGE, MUC and 5T4, or mutated or upregulated proteins such as p53 and growth receptors. In another embodiment, said foreign antigen comprise a microbial protein or a homologue thereof, preferably a human disease associated viral or bacterial protein, such as HPV, hepatitis, EBV or Helicobacter. In a preferred embodiment, said foreign antigen comprises a viral protein or a homologue thereof. Thus preferably, said mycobacterium is provided with a nucleic acid encoding said viral antigen or encoding a homologue thereof comprising at least 90% sequence identity with said viral protein. Preferably said viral protein is a human virus protein or an animal virus protein. Preferably a viral protein from a fish or cow pathogen. More preferably said virus comprises a Human Papilloma Virus (HPV), a hepatitis virus or an Epstein-Barr Virus (EBV).

The invention also provides a life, killed or attenuated mycobacterium of the invention. For immunization purposes it is preferred that a life or attenuated mycobacterium is used. Further provided is an immunogenic composition produced from a mycobacterium of the invention. In one embodiment of the invention said immunogenic composition further comprises a foreign antigen.

Provided is also the use of a mycobacterium of the invention for producing an immunogenic composition.

An immunogenic composition of the invention is preferably used for the immunisation of a human and/or a non-human animal. Preferably said non-human animal is a fish or a cow.

It has been found that presentation of particularly MHC-I peptides is enhanced when translocation of mycobacteria is promoted. The invention thus further provides a for enhancing and/or inducing an MHC-I type related immune response in an individual against a mycobacterial antigen, comprising providing said individual with a mycobacterium according to the invention, or an immunogenic composition according to the invention.

Further provided is the use of a nucleic acid encoding CFP10, ESAT6 and/or EspA to provide a mycobacterium with the capacity to translocate from a phagosome to the cytosol of a host cell, or to enhance said capacity.

In another aspect is provided the use of a nucleic acid encoding CFP10, ESAT6 and/or EspA to provide a mycobacterium with an enhanced capacity to induce and/or stimulate an MHC-I response in an individual provided therewith.

Further provided is the use of CFP10, ESAT6 and/or EspA for enhancing MHC-I type presentation of an antigen in an immunogenic composition, when provided to an individual.

The invention further provides a method for selecting a mycobacterium for the preparation of a vaccine comprising infecting cells permissive for said mycobacterium in vitro with said mycobacterium and determining whether said mycobacterium translocates to the cytosol of said infected cells. The invention further provides a method for selecting a mycobacterium for the preparation of a vaccine comprising infecting cells permissive for said mycobacterium in vitro with said collection and selecting from said collection a mycobacterium which translocates to the cytosol of infected cells. In this way different mycobacteria and differently manipulated mycobacteria can be pre-screened in vitro for their immunogenic potential in vivo, thus facilitating the generation vaccine compositions with enhanced immunogenicity, for instance when compared to vaccine compositions produced from non-translocating mycobacterial strains such as BCG. The invention further provides a method for obtaining an immune response in an individual comprising providing said individual with a mycobacterium according to the invention.

It is also possible to provide other bacteria with the property to translocate to the cytosol. The invention therefore further provides a method for enhancing and/or inducing cytosolic translocation of a bacterium comprising providing said bacterium with a nucleic acid for expression of CFP10, ESAT6 and/or EspA of a mycobacterium in said bacterium. Also provided is the use of a nucleic acid for expression of CFP10, ESAT6 and/or EspA of a mycobacterium in a bacterium for enhancing and/or inducing cytosolic translocation of said bacterium in a eukaryotic host cell. Enhanced and/or induced cytosolic translocation of such bacteria results an enhanced immune response of an individual when exposed to said bacterium. Thus the invention further provides a method for enhancing and/or inducing an MHC-I type related immune response in an individual against an antigen comprising providing a bacterium comprising said antigen with a nucleic acid for expression of CFP10, ESAT6 and/or EspA of a mycobacterium in said bacterium and administering said bacterium to said individual. In a preferred embodiment said bacterium is not a mycobacterium. Preferably said bacterium is a bacterium of a pathogenic bacterial species, preferably a legionella species, preferably l. pneumophila or a salmonella species. In another preferred embodiment said bacterium comprises N. gonorrhoeae or a S. aureus. Thus in a further aspect the invention provides a non mycobacterial bacterium comprising a nucleic acid for expression of CFP10, ESAT6 and/or EspA of a mycobacterium in said bacterium.

A vaccine composition can be administered in various ways. Cell-mediated immunity plays the principal role in containing infection, and the routes of vaccine administration and immunization influences immune response development. In infants, BCG vaccination is generally performed by subcutaneous immunization. This generally induces a Th1 cytokine response and stimulates cytotoxic T-lymphocyte activity in neonates. An alternative route of BCG administration, which does not induce the side effects associated with subcutaneous immunization, is via rectal delivery. This method induces a similar immune response and protection in several animal models without altering the recruitment patterns of activated T-cells. Intranasal immunization induces higher protection by rapid induction of IFN-γ and T-cell response in the lung tissue but there are some who have serious misgivings in using live bacilli. However, nasal administration of recombinant BCG as a means to deliver immune dominant antigens to the mucosa is possible.

In some application area's it is preferred to immunize via oral immunization. For instance, animals and particularly fish can be immunized with having to handle each individual animal separately. In a preferred embodiment the invention provides an animal food or a composition for the production of an animal food comprising a mycobacterium according to the invention, an immunogenic composition according to the invention or a bacterium according to the invention. Further provided is an oral vaccine comprising a mycobacterium or a bacterium of the invention.

The present invention shows that mycobacteria such as M. tuberculosis and M. leprae exist in two intracellular sites in human myeloid cells. Early, 2-48 h after infection, bacteria reside in a phagolysosome and at extended time points post infection, between 2 and 4 days for M. tuberculosis and between 4 and 7 days for M. leprae the bacilli translocate to the host cytosol. Bacteria in phagosomes rapidly colocalize with the late endosome and lysosomal markers CD63 and LAMP-1 and LAMP-2, which are delivered to the phagosome via fusion of multivesicular late endosomes or lysosomes within the first hours of infection. Confinement to the phagolysosome coincides with a period of static bacterial growth that is evident by quantitation of the number of bacteria per phagolysosome in each cell and CFU analysis. We also find that mycobacteria such as M. tuberculosis and M. leprae phagosomes lack transferrin receptor and early endosomal autoantigen 1 (EEA1) in DCs, extending the correlation between phagolysosomal localization and deficient growth. The invention thus further provides a method for infecting host cells with a mycobacterium comprising infecting host cells with said mycobacterium and determining after a period of at least 48 hours and preferably at least 72, more preferably 96 hours, the location of said mycobacterium in said host cells. This is preferably done using microscopy, however, other methods such as flow cytometric, or fractionation approaches are also within the scope of the invention.

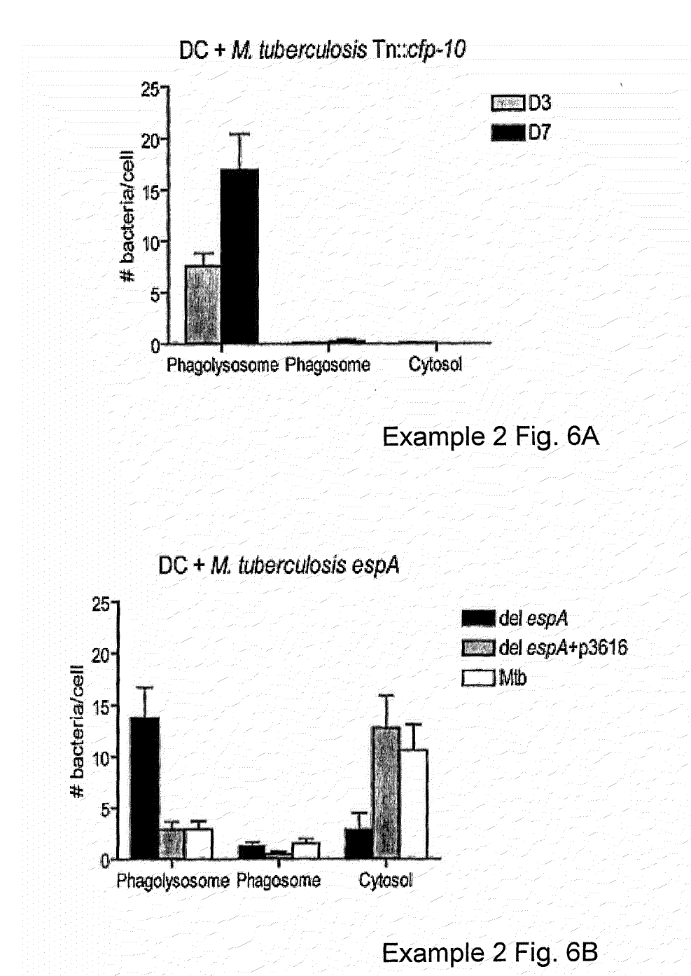

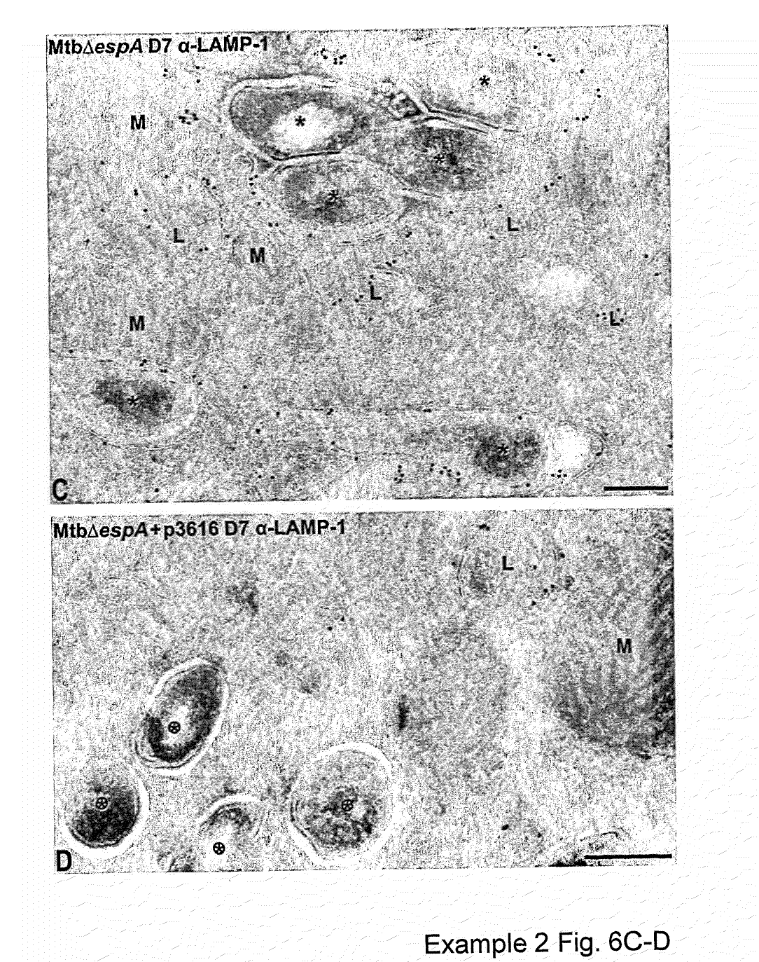

After several days of infection, M. tuberculosis and M. leprae are found in the host cytosol of human DCs and macrophages (FIG. 6). Previous studies showed evidence for cytosolic M. tuberculosis in several cell types including human pneumocytes, rabbit alveolar macrophages, and human monocytes (Myrvik et al., 1984; Leake et al., 1984), however, the prevailing paradigm has remained that M. tuberculosis reside in the endocytic system (Clemens and Horwitz, 1995; Russell, 2001; Russell et al., 2002; Orme, 2004; Vergne et al., 2004; Kang et al., 2005; Pizarro-Cerda and Cossart, 2006). Mycobacterium localization in infected macrophages has been extensively studied for over 40 years using an array of techniques and a number of Mycobacterium species as model organisms for M. tuberculosis. In general, the majority of these experimental systems only focused on the first 48 h following mycobacterium infection and were not always performed with virulent mycobacteria. Here we have used an extended time course to examine the localization of M. tuberculosis and M. leprae for up to 7d of infection. In our assays, the excellent preservation of cellular membranes in cryosections, coupled with immunological detection of endocytic markers allowed the quantitative assessment of mycobacterial localization to the cytosol at times beyond 2 days of infection.

Interestingly, phagolysosomal translocation coincides with an increase in M. tuberculosis titer that continues over the course of the infection. No cytosolic mycobacteria are found after DCs and macrophages phagocytose dead bacteria. Further, we find that the appearance of cytosolic bacteria requires the genes encoded in the ESX-1 region, and more specifically the secretion of CFP10 and ESAT6. This finding is further supported by the fact that BCG, which lacks a portion of the ESX-1 cluster called the RD1 region fails to translocate into the cytosol and remains localized to the phagolysosome. In addition to M. tuberculosis, the RD1 locus is also present in M. bovis, M. kansasii, M. marinum, M. africanum, and M. leprae (Berthet et al., 1998; Harboe et al., 1996). The ESX-1 region has an important role in the virulence of M. tuberculosis (Lewis et al., 2003; Hsu et al., 2003; Stanley et al., 2003). The genes encoded in the ESX-1 region are predicted to form a specialized secretory apparatus that secretes CFP10 and ESAT6. These proteins have an unknown function during infection, and are also potent T cell antigens recognized by both CD4+ and CD8+ T cells. EspA has an essential role in the secretion of CFP10 and ESAT6 (Fortune et al., 2005). Interestingly, the secretion of EspA also relies on CFP10 and ESAT6, as well as, the ESX-1 secretion system. The specific interactions formed between CFP10-ESAT6-EspA are not known, nor is it known if they function together upon secretion, but it has been suggested that one or more of these proteins may serve a chaperone function for the others (Fortune et al., 2005). Our analysis further implicates these important genes in translocation of M. tuberculosis from the phagolysosome and its replication in the cytosol.

Pathogens such as L. monocytogenes that lyse host phagosomes and replicate in the host cytosol induce potent CD8+ T cell responses. Lysis of the phagosomal membrane requires the cholesterol dependent cytolysin Listeriolysin O (LLO), which has a slightly acidic pH optimum and a short-half life in the host cytosol (Glomski et al., 2002; Schnupf et al., 2006; Decatur and Portnoy, 2000). The multiple levels of regulation of LLO compartmentalizes its activity to function in the lysis of the phagosomal membrane, but not the host plasma membrane, and mutants that fail to do so are avirulent in mouse models of infection (Glomski et al., 2003). Along these lines it is interesting to speculate that an analogous mechanism may function during M. tuberculosis infection. The intracellular expression of CFP10-ESAT6-EspA clearly follows infection of human macrophages. Guinn et al. have reported that M. tuberculosis lyses host cells and spreads to uninfected macrophages over a 7d time course, and that this occurs in an RD1-dependent manner (Guinn et al., 2004b). Recently, M. marinum has been shown to escape from phagosomes in infected macrophages and spread to neighbouring cells via actin based motility (Stamm et al., 2003; Stamm et al., 2005). It is noteworthy that in a Rana pipiens model of long-term granuloma formation, 60% of M. marium phagosomes were fused with lysosomes (Bouley et al., 2001). Therefore, it seems likely that M. tuberculosis has evolved additional mechanisms of immune escape that allow survival when the blockade of phagosome-lysosome fusion is overcome by the host. These might be significant at later stage of infection or upon cytokine activation of infected antigen presenting cells.

The immune response to M. tuberculosis is a dynamic process involving both CD4+ and CD8+ T cells (Flynn and Chan, 2001), which predominate as the major INFγ secreting cells at different stages of infection: CD4+ T cells dominate during acute infection and CD8+ T cells during persistent infection (Lazarevic et al., 2005). How antigens from intracellular bacteria gain access to the MHC Class I antigen loading pathway in the ER remains an intense area of study. Several groups have suggested direct fusion between the ER and phagosome during phagocytosis (Houde et al., 2003; Ackerman et al., 2003; Guermonprez et al., 2003), however, quantitative assessment of ER markers on both model latex bead phagosomes and M. avium containing phagosomes contradict those findings (Touret et al., 2005). Similarly, we find no evidence for the localization of ER markers to the Mycobacterial phagosome after infection, but rather we suggest that M. tuberculosis and M. leprae antigens presented by MHC Class I are most likely derived from bacteria that have entered the host cytosol as shown here.

It is significant that BCG, which is used worldwide as a mycobacterial vaccine strain remains restricted to the phagolysosome following infection of DCs and macrophages, whereas virulent M. tuberculosis does not (FIG. 6). BCG vaccination has questionable efficacy against the highly infectious pulmonary form of tuberculosis, and it fails to generate a strong MHC class I restricted T cell response. The work presented here emphasizes that non-virulent mycobacterial species fail to translocation the phagosome and suggests this may account for their poor capacity to stimulate critical CD8+ T cell responses. Interestingly, innovative vaccine approaches have genetically engineered BCG to express LLO as a mechanism to generate more potent MHC Class I-restricted responses. Indeed, LLO+BCG are more effective vaccines than the isogenic BCG parental strain (Grode et al., 2005). Designing vaccines that mimic virulent strains in translocating into the cytosol is likely to be a critical step forward in producing more effective vaccines for tuberculosis.

### EXAMPLES

### Example 1

### Results

### M. Tuberculosis and M. Leprae Reside in a Phagolysosome Early After Phagocytosis

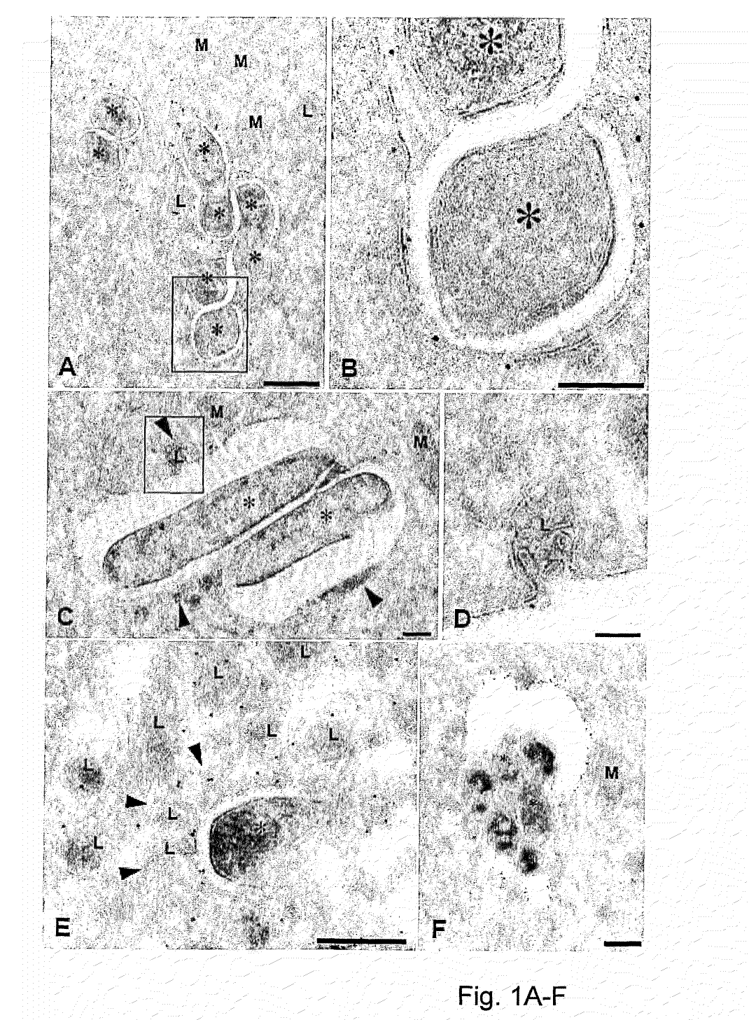

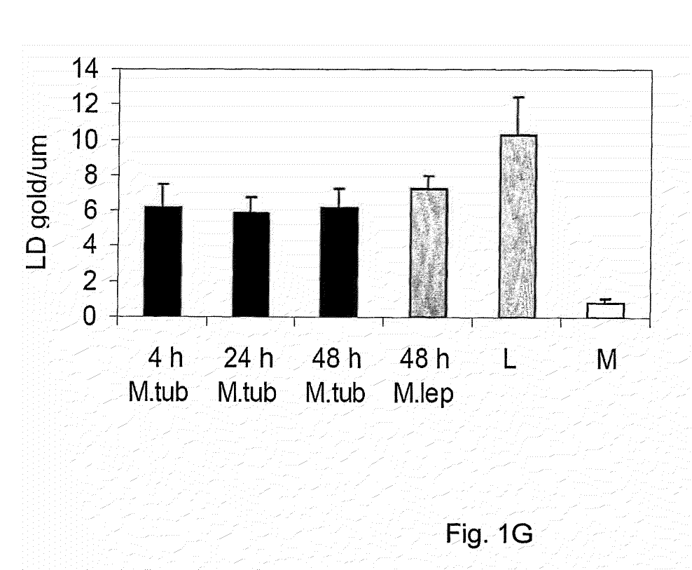

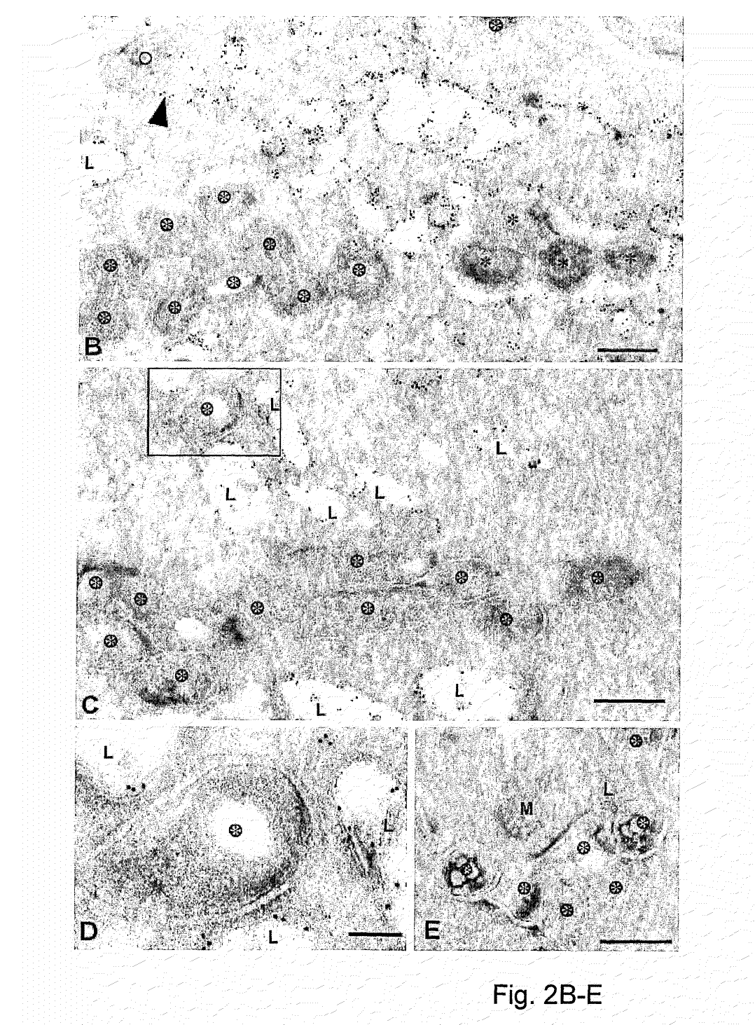

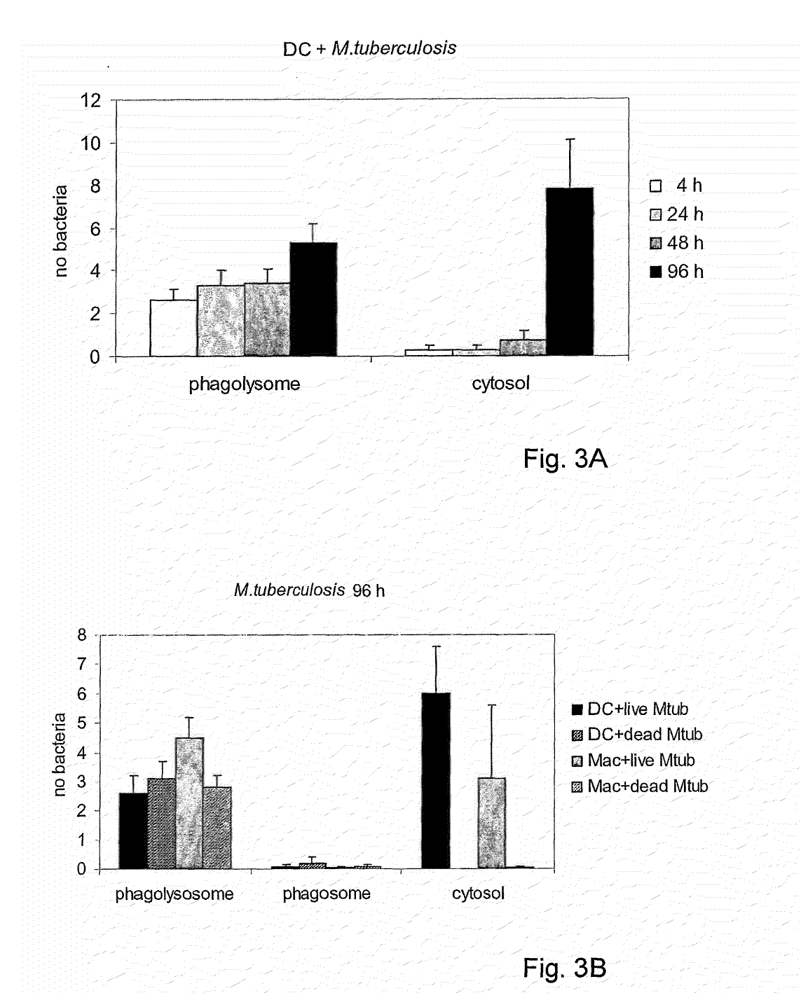

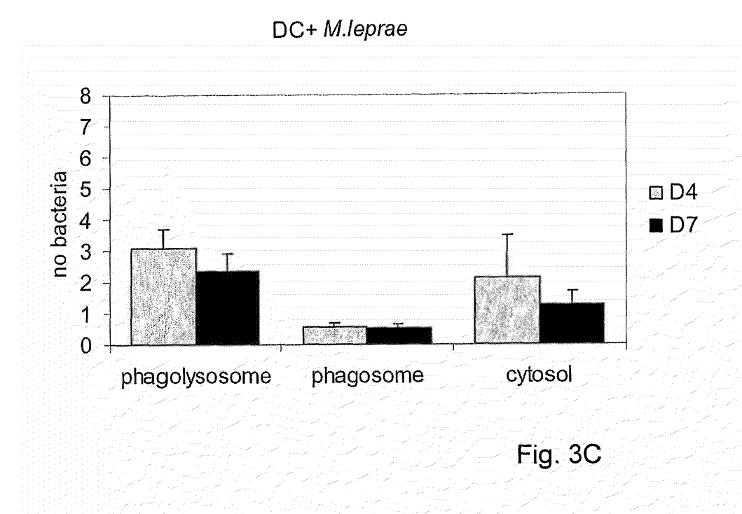

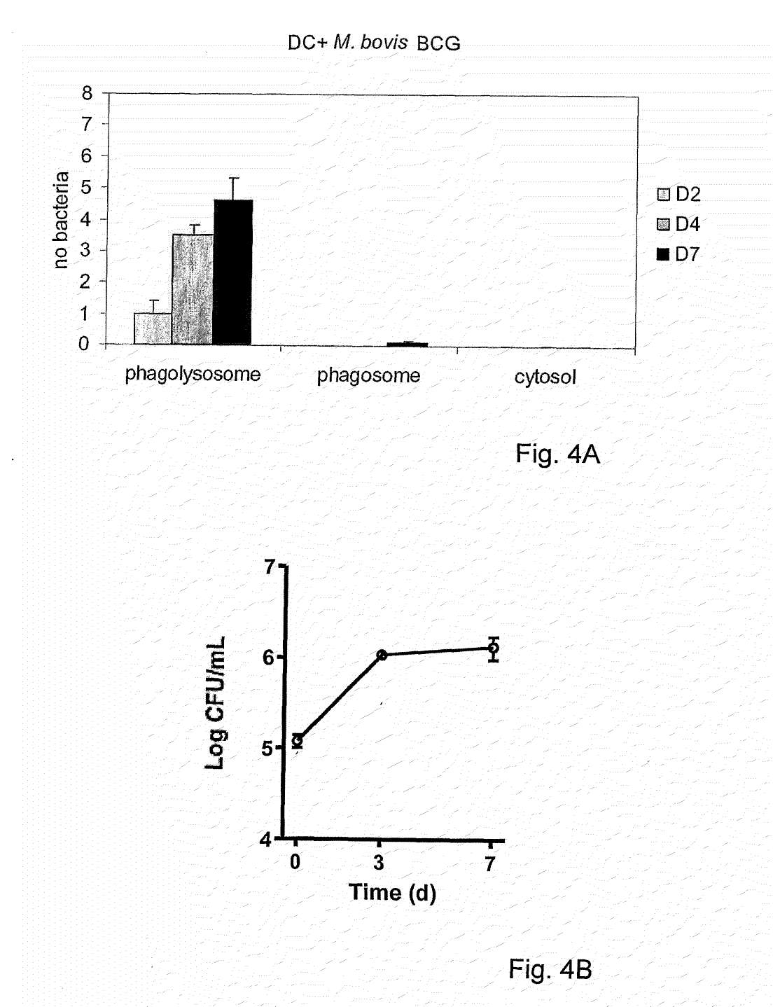

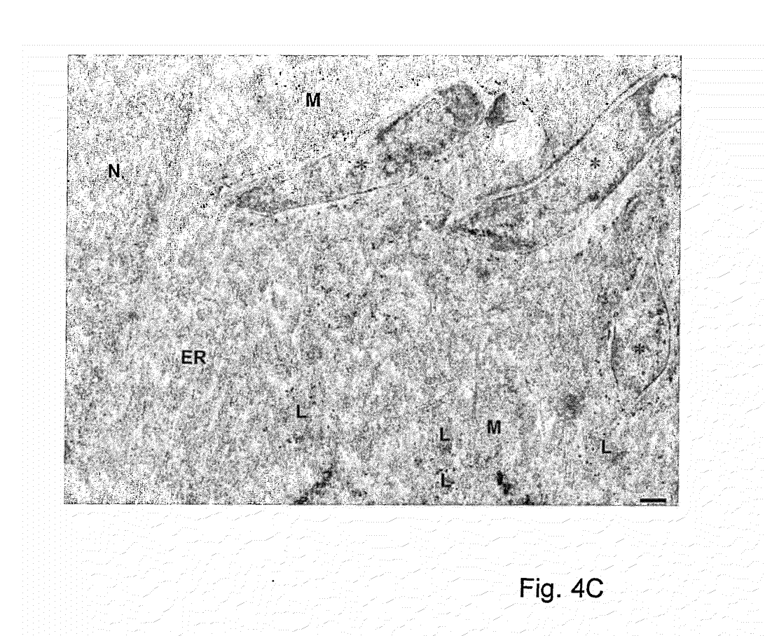

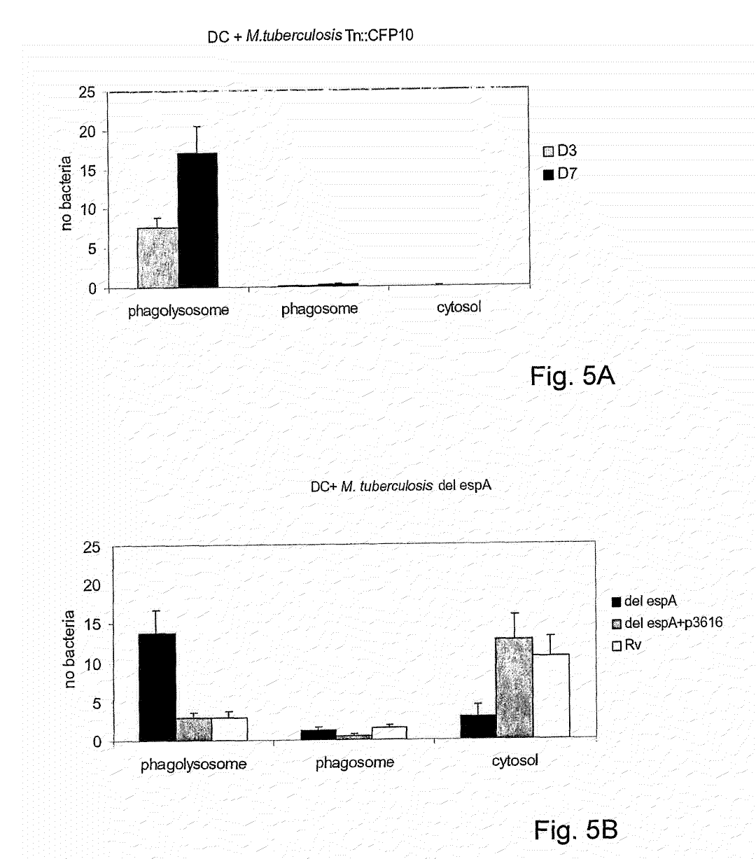

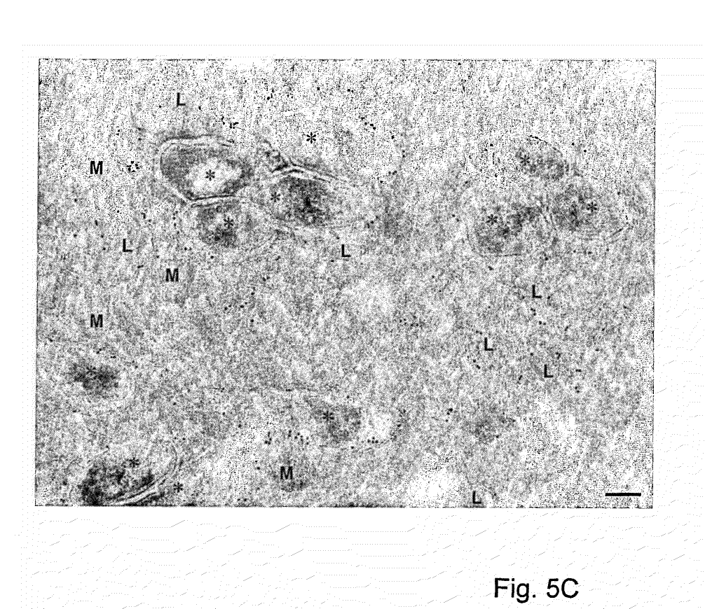

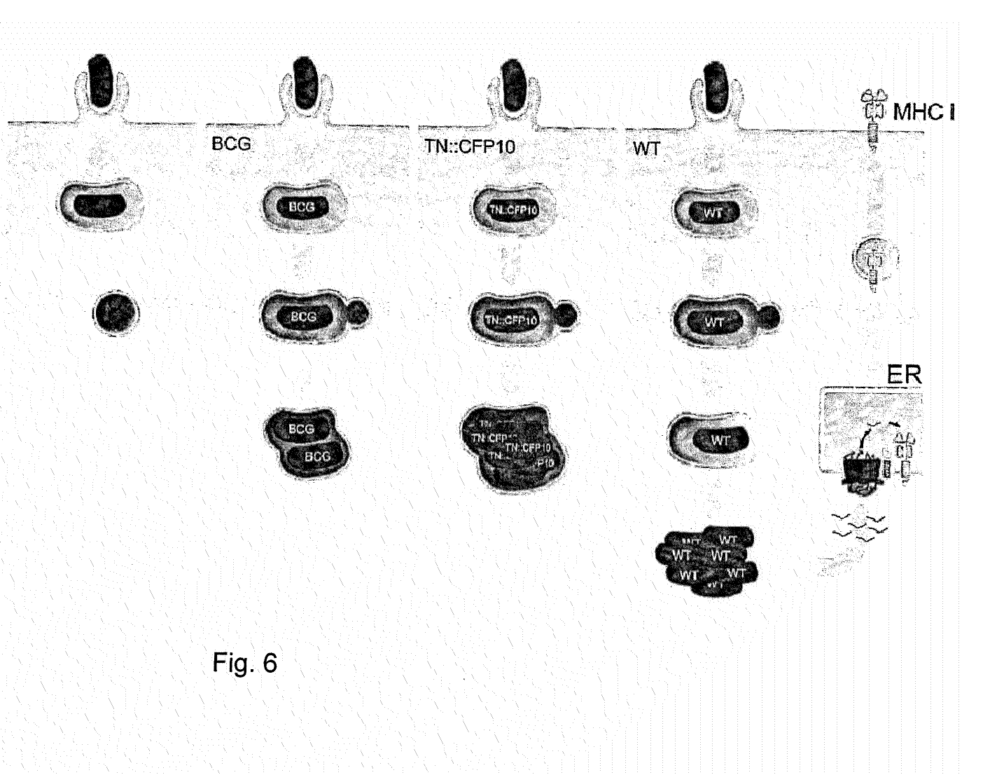

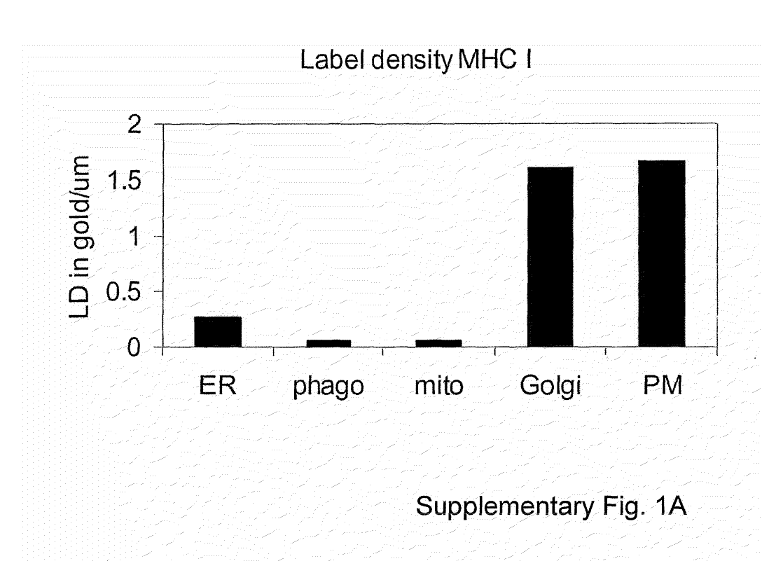

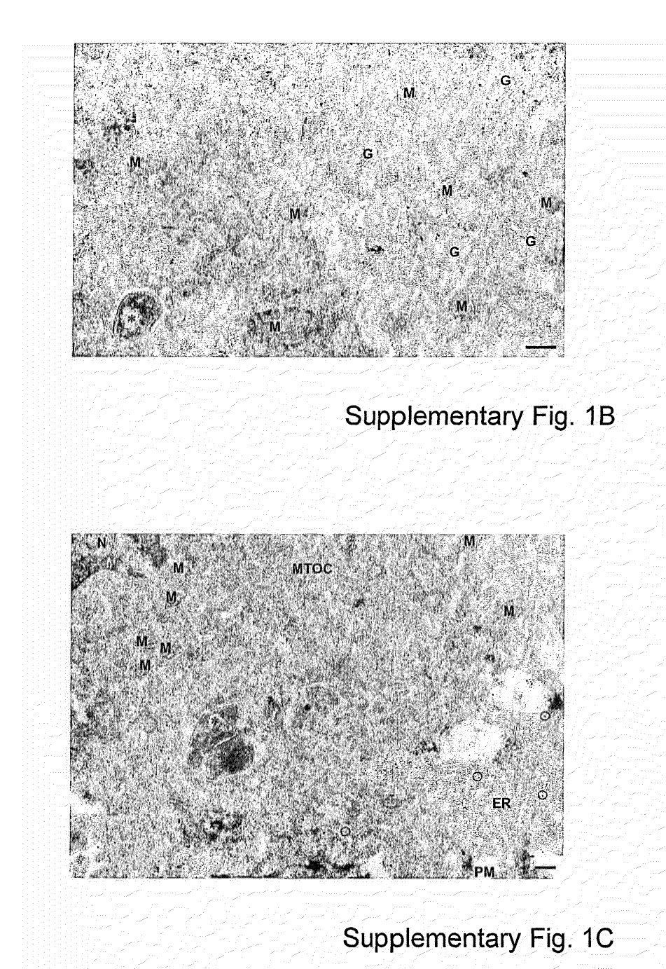

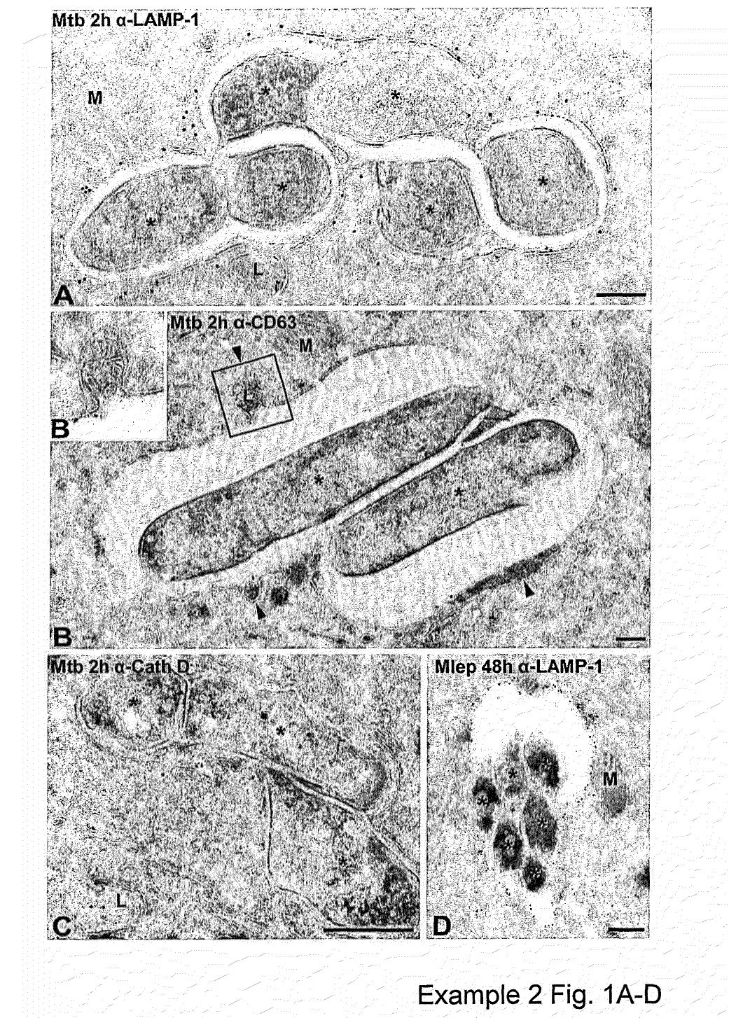

The subcellular localization of M. tuberculosis and M. leprae was analyzed in freshly isolated human monocyte-derived DCs. Monocyte-derived DCs were differentiated from human CD14+ monocytes precursors for 5 days in GMCSF and IL-4, and subsequently infected with M. tuberculosis or M. leprae. Samples were fixed at various times after infection (8 min to 48 h) and processed for cryo-immunogold electron microscopy. We analyzed the localization of early and late endosomal markers to the M. tuberculosis or M. leprae phagosome. Two hours after infection, the phagosome lacked the early endosomal markers transferrin receptor (TfR) and early endosomal autoantigen 1 (EEA1), which instead were exclusively localized to early endocytic and recycling endosome membranes (Table 1). The phagosome was also negative for the late endosomal cation-independent mannose 6-phosphate receptor (Table 1). In contrast, both M. tuberculosis and M. leprae phagosomal membranes stained positively for the lysosomal associated membrane proteins LAMP-1, LAMP-2, and CD63 (FIG. 1A-F and Table 1). In immature DCs, these makers differentially localize in multilamellar and multivesicular vesicles such as the MHC class II compartment (MIIC) (Peters et al., 1991), with LAMP-1 and LAMP-2 localized on the limiting membrane and CD63 on internal membranes. Following the maturation of DCs, the multivesicular nature of MIICs is modified and all three markers localize to the limiting membrane of the mature DC lysosome (MDL) (van der Wel et al., 2003). The efficient delivery of these molecules to the phagosome following infection was visualized by the direct fusion of multivesicular lysosomes with the phagosome (FIGS. 1C, D and E arrow heads).

The fusion of lysosomes with the M. tuberculosis phagosome at early time points led us to investigate if LAMP-1 accumulated on phagosomes over time. Over the course of 48 hours of infection, the average labelling density of LAMP-1 on M. tuberculosis (FIG. 1E) and M. leprae (FIG. 1F) phagosomes remained stable and had levels that were only slightly lower than the lysosomal membranes monitored in the same cells (FIG. 1G). Thus, following the infection human monocyte-derived DCs, the mycobacteria reside in a compartment that readily fuses with lysosomes.

To determine if the ER contributed to the phagocytosis of either microbe, immunogold labelling was performed on thawed cryosections against MHC Class I and two ER resident proteins: the MHC class I peptide transporter TAP and PDI, a soluble ER protein. None of these molecules were detected on M. tuberculosis or M. leprae membranes at multiple time points (Table 1 and Supplementary FIG. 1). Quantification of the MHC class I labelling density in the ER and on the phagosomal membrane demonstrated that the levels in the phagosome do not rise above background levels of staining detected in mitochondria (Supplementary FIG. 1). Furthermore, despite the close proximity of ER cisternae to the phagosomal membrane, fusion between the membranes was not detected (n=300).

### M. Tuberculosis Access the Host Cytosol and Replicate

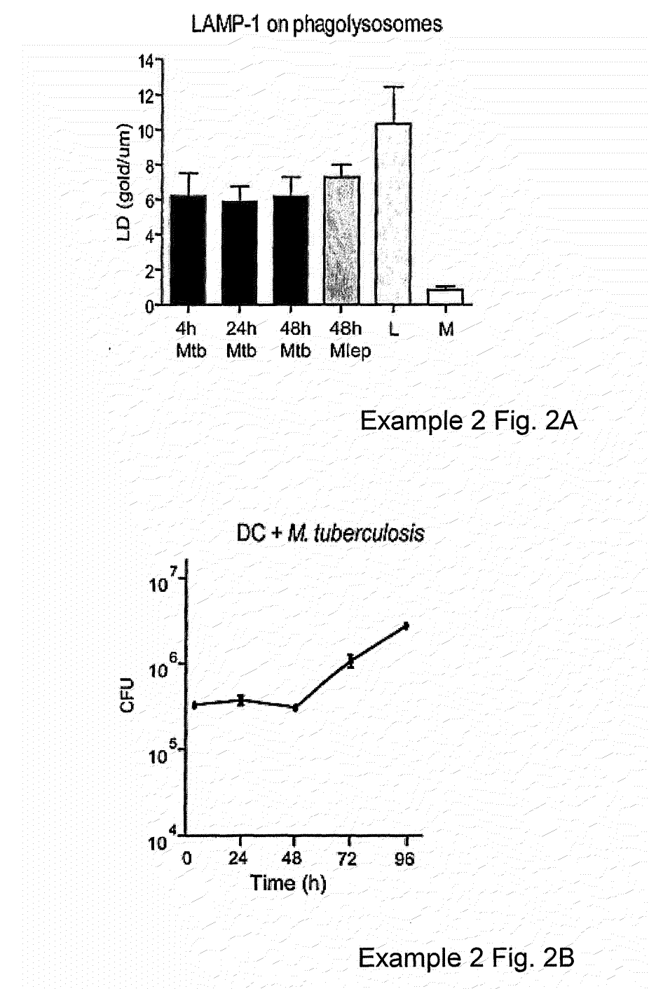

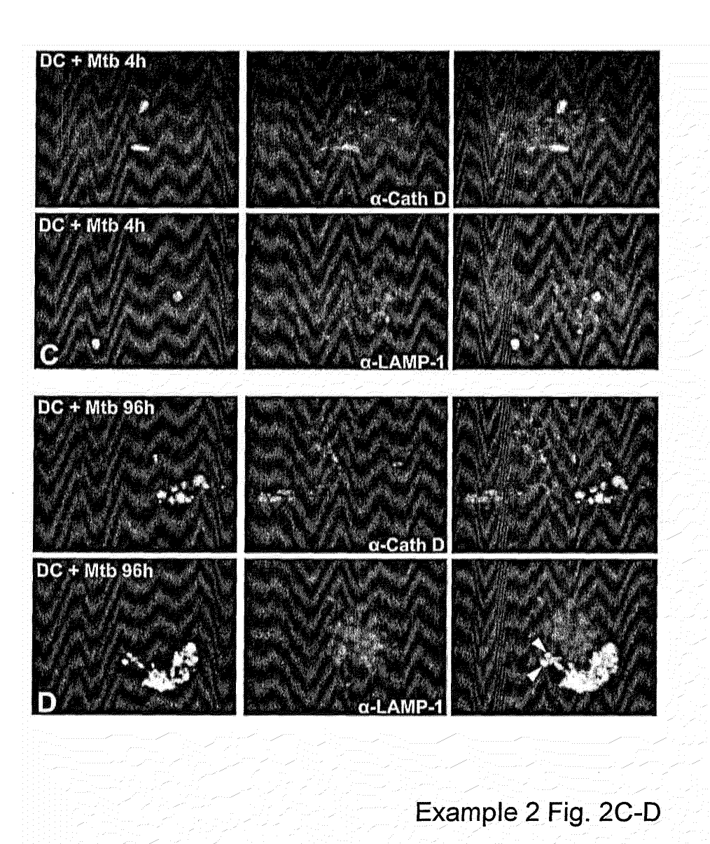

It is thought that in macrophages, the access of the phagosome to the early endocytic system enables M. tuberculosis and M. leprae to evade acidification and degradation, and also permits growth by allowing extracellular nutrients to reach replicating bacteria. The localization of M. tuberculosis to a phagolysosomal compartment in monocyte-derived DCs led us to investigate the intracellular survival and growth following infection in these cells. Monocyte-derived DCs were infected with M. tuberculosis and plated in replicate wells of a 24-well plate. At each time point, DCs were lysed and the number of colony forming units (CFU) per well was enumerated. During the initial 48 h of infection, the titer of M. tuberculosis remained constant indicating no net growth in monocyte-derived DC culture over this time (FIG. 2A). Throughout this time period, M. tuberculosis were found exclusively in phagolysosome, as shown above (FIG. 1).

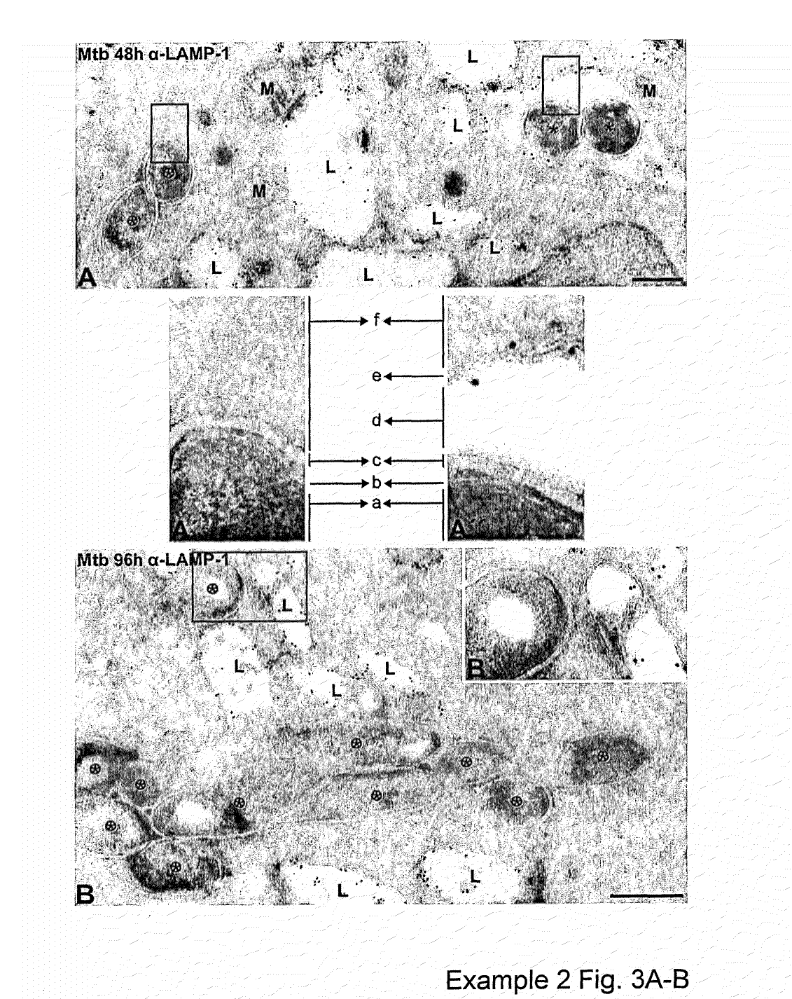

The static growth kinetics of M. tuberculosis and the failure of early endocytic vesicles to reach the phagolysosome during the first 48 h of infection indicate that the phagolysosomal compartment restricts bacterial replication. However, following the first 48 h period, the titer of M. tuberculosis increased steadily over the next 48 h of culture (FIG. 2A). In subsequent experiment, similar growth kinetics were observed and the bacterial titer continued to increase between 3 and 7d post-infection. Thus, M. tuberculosis persist during the initial 48 h infection period in monocyte-derived DCs, but are able to replicate significantly only after that time point. The increase in bacterial titer between day 2 and 3 suggested that alterations occur to the phagolysosome that create a more favourable growth environment. To investigate the intracellular localization of the bacteria in this timeframe, monocyte-derived DCs infected with M. tuberculosis were fixed and processed for cryo immuno-gold labelling with anti-LAMP-1 antibody at 48 and 96 h. As at the earlier time points, M. tuberculosis primarily localized to LAMP-1 positive phagolysosome at 48 h after infection, and bacteria that resided in LAMP-1 negative vesicles was negligible (n=500; FIG. 2B). Occasionally, bacteria were found that lacked the characteristic electron lucent zone (Armstrong and Hart, 1971) and did not stain positively for LAMP-1 (FIG. 2B). Importantly, these bacteria were not present in membrane enclosed compartments and appeared to be localized to the cytosol. In some instances, bacteria only partially surrounded by phagolysosomal membranes were seen and may represent bacteria at an intermediate stage of translocation from the phagolysosome (FIG. 2B arrowhead). Strikingly, inspection of cells infected for 96 h revealed that the percentage of cytosolic M. tuberculosis increased with a function of time and that large clusters of cytosolic bacteria were observed (FIGS. 2C, D; FIG. 3). High magnification images of individual bacteria confirmed that these bacteria lacked phagolysosomal membranes despite residing in close proximity to LAMP-1 positive lysosomes (FIG. 2D). From these images, we concluded that at later stages after infection a subset of intracellular M. tuberculosis reside in the cytosol of the host cell rather than in a membrane enclosed phagolysosome. To determine if the appearance and large clusters of cytosolic bacteria could be associated with growth of M. tuberculosis, the number of phagolysosomal bacteria and cytosolic bacteria were quantified over time. The number of cytosolic M. tuberculosis per cell rose sharply between 2d and 4d, increasing approximately 10-fold, while the number of phagolysosomal bacteria increased at a much slower rate (FIG. 3A). Likewise, larger clusters of M. tuberculosis were observed in the cytosol than in phagolysosomes. In no instances did we observe LAMP-1 in the absence of phagosomal membrane, confirming our ability to observe membranes surrounding the bacteria. Similar observations were made in M. tuberculosis infected human monocyte derived macrophages (FIG. 3B) and THP1 cells (not shown) after 4d, as well as, in M. leprae infected monocyte derived DCs examined at 4 and 7 days after infection (FIGS. 2E and 3C). In the M. leprae infected cells, relatively small clusters of cytosolic bacteria were observed, which slightly decreased in size between 4 and 7 days. The decreasing numbers should be attributed to the well known disability of M. leprae to multiply in cultured cells However importantly, both M. tuberculosis and M. leprae enter the host cytosol and M. tuberculosis increases in number over time.

To determine if phagolysosomal translocation required an active process of mycobacteria, we examined the localization of heat-killed M. tuberculosis in monocyte derived DCs and macrophages. In all cell types, heat-killed M. tuberculosis resided exclusively in phagosomes and phagolysosomes that stained positively for LAMP-1 (FIG. 3B). It is noteworthy that the number of heat killed bacteria per phagolysosome is comparable to the number of phagosomal bacteria in the live infection, indicating that bacterial burden alone in the phagosome is not sufficient for the cytosolic phenotype.

### Phagosome Translocation Requires the RD1 Region

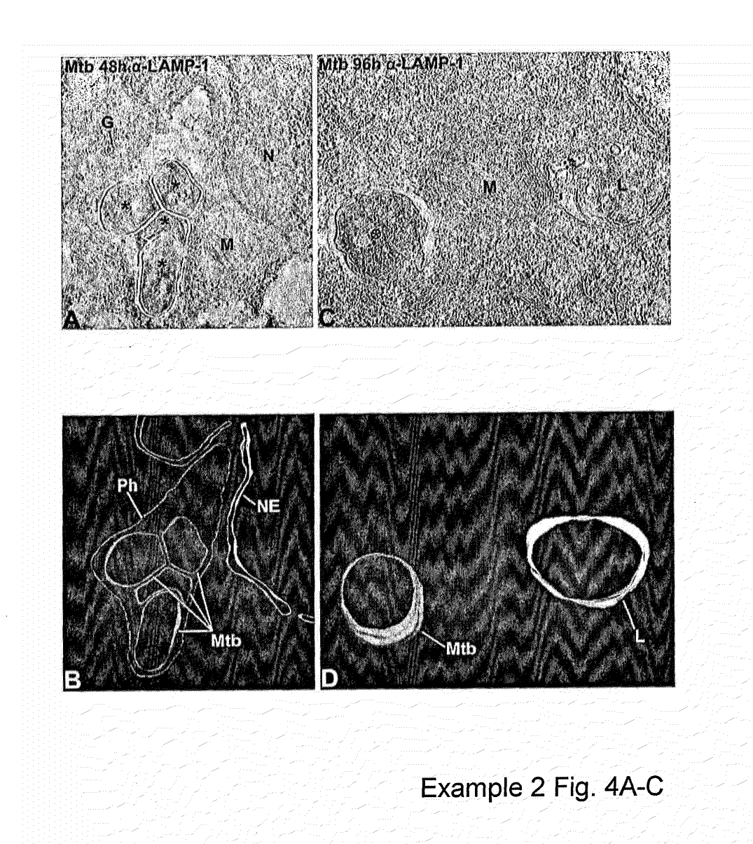

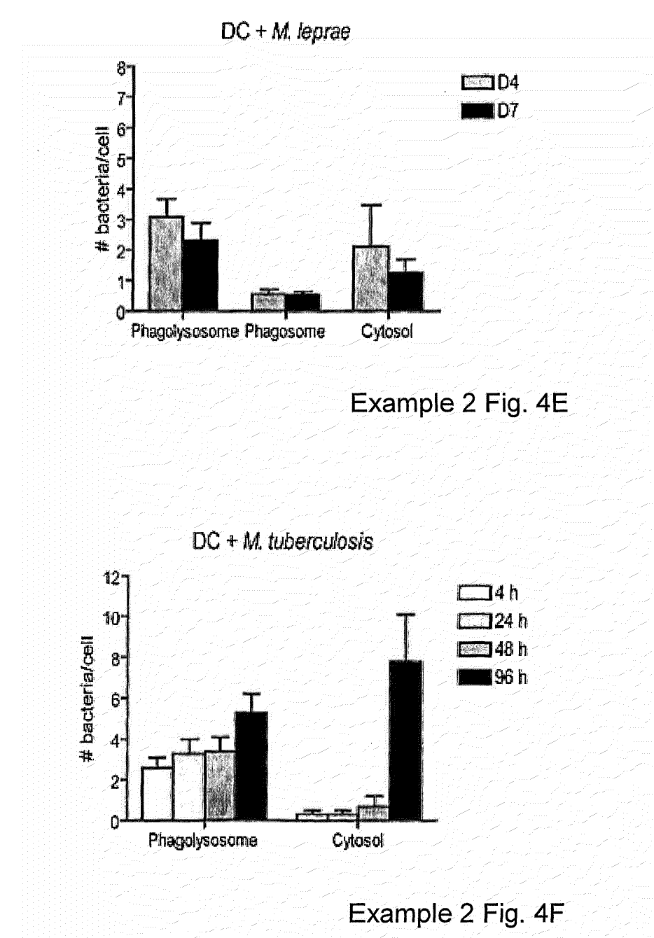

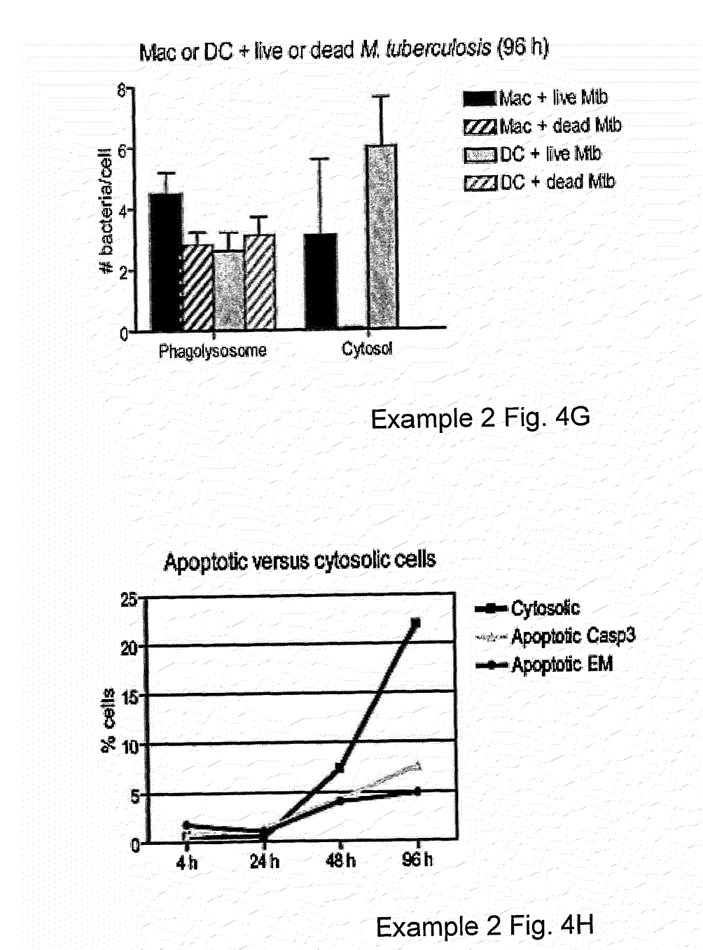

The observation that phagosome translocation required live M. tuberculosis led us to investigate if only fully virulent bacteria access the cytosol. To address this, the intracellular localization of the widely used vaccine strain M. bovis BCG (Pasteur strain) was examined and compared to virulent M. tuberculosis H37Rv. Human monocyte derived DCs infected with BCG were investigated at various days after infection. Strikingly, BCG was confined to LAMP-1 positive membrane enclosed compartments at all three time points (2, 4, and 7 d) studied and no cytosolic mycobacteria were detected in these samples (FIG. 4). In addition, the ultrastructure of the phagolysosomes enclosing BCG lacked the electron lucent zone between the phagosomal membrane and the cell wall that is characteristic of M. tuberculosis phagolysosomes, suggesting that these bacteria differentially modulate their intracellular environment. Although BCG failed to enter the cytosol, the number of phagolysosomal BCG increased over time and in a subsequent experiment the titer of BCG increased over time (FIG. 4B). As with the dead bacteria, this reinforces that access to the cytosol does not occur simply by mycobacteria out growing its phagosomal space.

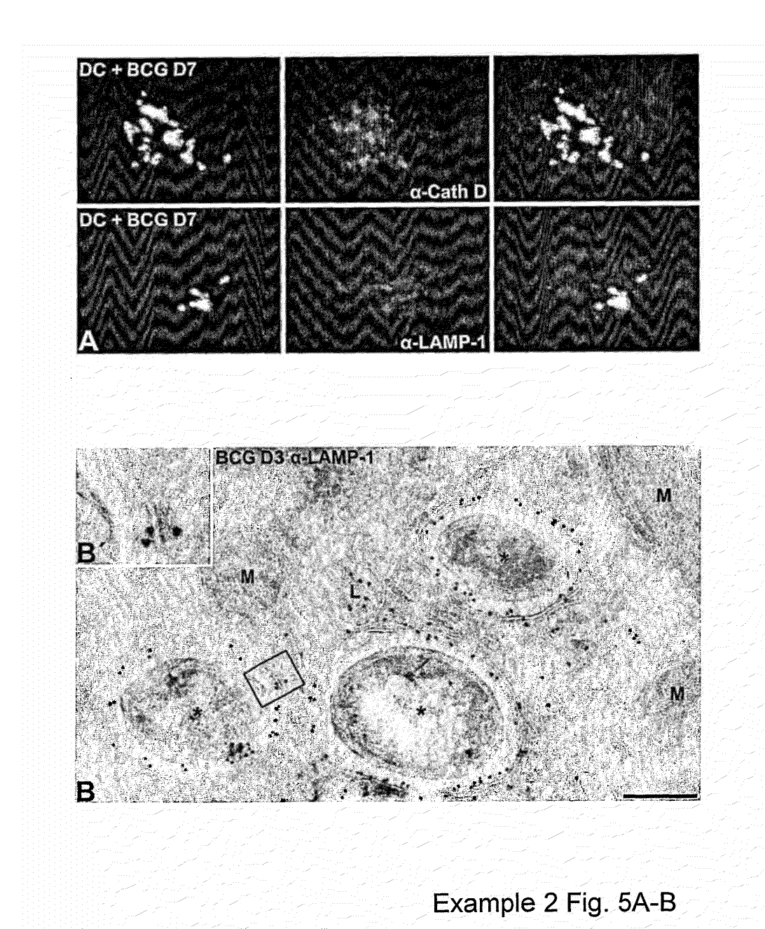

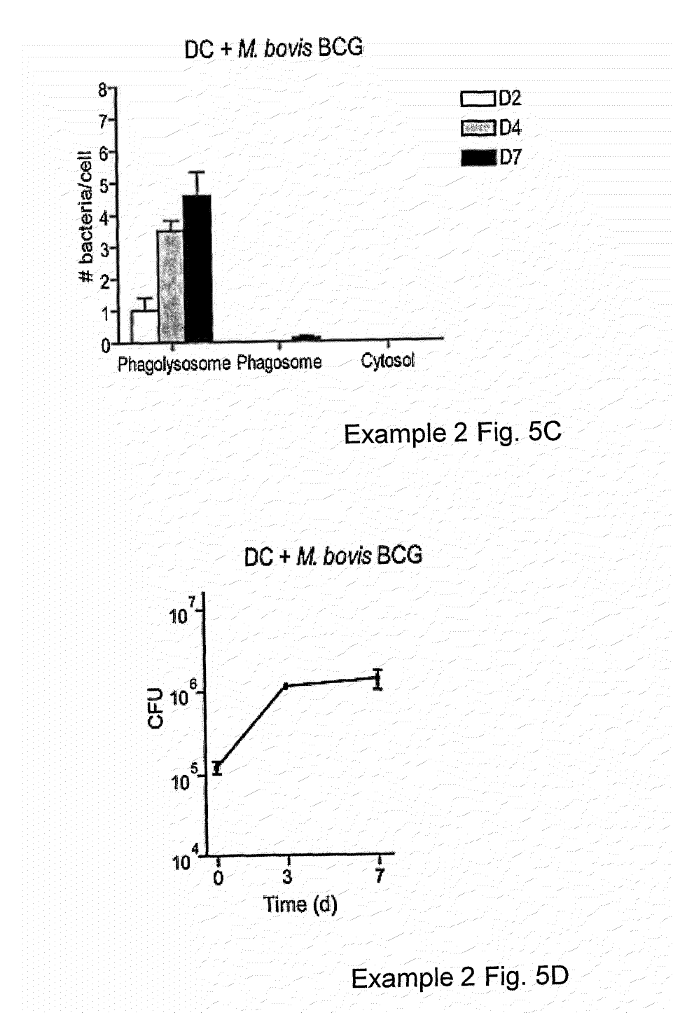

Dissection of the genetic differences between M. tuberculosis and BCG identified several large deletions from BCG that are present in M. tuberculosis and M. leprae (Harboe et al., 1996; Gordon et al., 1999; Behr et al., 1999; Philipp et al., 1996). From these 16 regions of difference (RD1-16) only RD1 is absent from all BCG strains thus far tested (Mostowy et al., 2002; Tekaia et al., 1999; Brosch et al., 2002). RD1 is part of a 15-gene locus known as ESX-1 that encodes a specialized secretion system dedicated to the secretion of CFP10 and ESAT6. In addition to the genes encoded in ESX-1, a second unlinked locus encoding espA is required for CFP10 and ESAT6 secretion (Fortune et al., 2005). The deletion of RD1 in BCG and the importance of the ESX-1 secretion system in virulence (Brodin et al., 2006) led us to test whether CFP10 and ESAT6 were required for M. tuberculosis access to the cytosol. This was first examined by using a M. tuberculosis strain containing a transposon insertion in cfp 10 (Rv3874), which prevents the synthesis of CFP10 and ESAT6 (Guinn et al., 2004b). Like BCG, this mutant failed to enter the host cytosol over the course of a 7d infection and resided in LAMP1+ compartments (FIG. 5A). Next, we used a □espA strain of M. tuberculosis to determine if the secretion of CFP10 and ESAT6 were required for the cytosolic phenotype. Following infection of monocyte derived DCs, the □espA strain and the □espA strain carrying the empty complementing vector (□espA pJEB) localized to LAMP-1 positive compartments and few bacteria were detected in host cytosol (FIGS. 5B, C). Strikingly, complementation of espA restored the number of cytosolic bacteria to a similar level as wild-type M. tuberculosis (FIG. 5B), demonstrating a role for the ESX-1 system and the secretion of CFP10 and ESAT6 in the translocation of M. tuberculosis from the host endocytic system.

### Material and Methods

### Human Cell Cultures

Peripheral blood mononuclear cells (PBMC) were isolated from healthy human donors as previously described (Porcelli et al., 1992). CD 14+ monocytes were positively selected from PBMC using CD14 microbeads and magnetic cell separation (Miltenyi Biotee, Auburn Calif.). Immature monocyte-derived DCs were prepared from CD14+ monocytes by culture in 300 U/ml of granulocyte-macrophage colony-stimulating factor (GM-CSF, Sargramostim, Immunex, Seattle, Wash.) and 200 U/ml of IL-4 (PeproTech, Rocky Hill, N.J.) for 5d in complete RPMI medium (10% heat-inactivated FCS/20 mM Hepes/2 mM L-glutamine/1 mM sodium pyruvate/55 μM 2-mercaptoethanol/Essential and non-essential amino acids). GMCSF and IL4 were replenished on d2, d5, and d9 after isolation. Macrophages were prepared by culture of CD14+ monocytes in IMDM with 10% human AB serum, 2 mM L-glutamine, and 50 ng/mL M-CSF (PeproTech, Rocky Hill, N.J.).

### Mycobacterial Infections

M. tuberculosis strains were grown to mid-logarithmic phase from frozen stocks in 7H9 Middlebrook media containing OADC enrichment solution and 0.05% Tween-20 for 1 week at 37° C. The wild-type M. tuberculosis strain used in these studies was H37Rv expressing green fluorescent protein (GFP) (Ramakrishnan et al., 2000). The BCG Pasteur strain was provided by Barry Bloom. The Tn::Rv3874 (cfp10) and the □espA strain have been previously described (Guinn et al., 2004a; Fortune et al., 2005). The □espA strain complemented strain encodes espA under the control of its native promoter on an integrating vector. The construct has been shown to complement the □espA mutation for ESAT6 secretion (S. Fortune, Personal communication). M. leprae were purified from mouse footpads as previously described and used in experiments one day after isolation (Adams et al., 2002). For in vitro infections, bacteria were harvested and suspended in RPMI containing 10% FCS, 2% human serum and 0.05% Tween 80, followed by washing in RPMI complete media. Cultures were filtered though a 5 μM syringe filter to obtain cell suspensions and counted using a Petroff-Houser chamber. Bacteria were added to DC and macrophage cultures at an MOI˜10 and plates were centrifuged for 2 min at 700 rpm prior to incubation at 37° C. with 5% CO₂. After 1 h, infected macrophage cultures were washed three times with warm culture media to remove free mycobacteria. For DC cultures, media was removed after 4 h of infection, diluted ˜1:6 in prewarmed RPMI complete media, centrifuged at 1000 rpm for 2 min, and resuspended in RPMI complete media supplemented with GMCSF/IL4. Culture wells were washed with RPMI three times to remove any remaining extracellular bacteria prior to replating DCs.

Colony forming units (CFU) were enumerated by lysing infected antigen presenting cells in sterile water with 0.1% saponin for 5 min. Lysed cells were repeatedly mixed and dilutions were made in sterile saline containing Tween-20. Diluted samples were plated on 7H11 Middlebrook agar plates (Remel) and colonies enumerated after 2-3 weeks of growth.

### Electron Microscopy

At each time point, cells were fixed by adding an equal volume of 2× fixative (0.2M PHEM buffer and 4% paraformaldehyde) to plates immediately after removal from the incubator. Cells were fixed for 20-24 h at room temperature and recovered using a cell scraper. Fixed cells were stored in 0.1M PHEM buffer and 0.5% paraformaldehyde until analysis. Fixed cells were collected, embedded in gelatine, cryosectioned with a Leica FCS and immuno labelled as described previously (Peters et al., 2006). Samples were trimmed using a diamond Cryotrim knife at −100° C. (Diatome, Switzerland) and ultrathin sections of 50 nm were cut at −120° C. using an Cryoimmuno knife (Diatome, Switserland). Immuno-gold labelling was performed using lysosome associated membrane protein 1 and 2 (LAMP-1 and LAMP-2 clone H4A3 and H4B4 from BD Biosciences), CD63 (M1544 Sanquin the Netherlands), mannose 6 phosphate receptor (M6PR a gift from Dr. V. Hsu), EEA1, Transduction labs Lexington, Ky.), Transferrin receptor (TfR H68.4 (CD71) Zymed), MHC class I (HC10 a gift from Dr. J. Neefjes), TAP (198.3 a gift from Dr. J. Neefjes) and PDI (a gift from Dr. H. Ploegh). Antibodies were labelled with rabbit anti-mouse bridging serum (DAKO) and protein-A conjugated to 10 nm gold (EM laboratory, Utrecht University). Sections were examined using a FEI Tecnai 12 transmission electron microscope.

### Brief Description of the Drawings of Example 1

### FIG. 1

In early stages of infection, M. tuberculosis and M. leprae reside in LAMP-1 containing phagolysosomes.

(A) Immunogold labelling against LAMP-1 on cryosections of a DC infected with M. tuberculosis for 2 hours.
(B) Enlargement of (A) showing that on the limiting membrane the phagolysosomes are immunogold labelled with LAMP-1.
(C) CD63 labelling on the limiting membrane of the phagolysosome in a DC infected with M. tuberculosis for 2 hours. In addition to labelling with these lysosomal markers several fusion events of lysosomes with the phagolysosome are detected (arrowheads). Note the electron lucent zone between the phagosomal membrane and the bacterial cell wall.
(D) Enlarged image of fusion between (multi-vesicular) lysosome and the phagolysosome.
(E) Later in the infection of M. tuberculosis (48 hours), mature DC lysosomes (MDLs) fuse with the phagosomal membrane.
(F) Labelling of LAMP-1 on cryosections of DC infected with M. leprae for 48 hours.
(G) The LAMP-1 labelling density: number of gold particles per μm phagosomal membrane (LD) as determined on at least 30 phagolysosomes in DCs infected with M. tuberculosis for 2, 24, 48 hours, and 48 h (M. leprae is included for the last time point) and compared to the LD on the limiting membrane of lysosomes or the background labelling on mitochondria in the same cells.
Asterisks indicate mycobacteria in phagolysosomes, M: mitochondria, L: lysosomes, arrowheads: fusion profiles.

### Bar: A) 500 nm, B, C) 200 nm, D) 100 nm, E, F) 300 nm

### FIG. 2

The relative amount of M. tuberculosis in DCs increases after 48 hours of infection, which coincides with translocation from the phagolysosome.

(A) The colony forming units (CFU) determined for M. tuberculosis infected DCs. Multiple experiments from which a representative figure is shown, all demonstrated that the CFU increased after 48 hours, suggesting that replication was significantly (small error bars) initiated after 48 hours of infection.
(B) Electron micrograph of a DC infected with M. tuberculosis for 48 hours showing two different subcellular locations: one the mycobacteria are observed in membrane enclosed phagolysosomes (asterisk) which are characterized by an electron lucent zone between the phagosomal membrane and the bacterial cell wall and immunogold labelling with LAMP-1 on the phagosomal membrane. The second subcellular location of mycobacteria is in the cytosol (encircled asterisk). These mycobacteria lack the enclosure of a membrane, the LAMP-1 labelling and the lucent zone. Occasionally, intermediate stages are detectable from which the LAMP-1 positive phagolysosomal membrane appears to retract from the mycobacterium (circle and arrow head).
(C) Clusters of M. tuberculosis present in the cytosol are abundant in live DCs infected for 96 hours.
(D) Enlarged image of (C) showing LAMP-1 positive limiting membranes of lysosomes and small vesicles however, such bi-layered membrane profiles are absent around the mycobacteria.
(E) Electron micrograph of a monocyte derived DC infected with M. leprae for 4 days showing a cytosolic location.
L: lysosomes, M: mitochandria, asterisk: mycobacteria in phagolysosomes, encircled asterisks: cytosolic mycobacteria, circle: intermediate stages of mycobacteria retracting from phagolysosome and bar: B, C, E) 500 nm, in D) 100 nm.

### FIG. 3

Number of live M. tuberculosis increase in the cytosol of live DCs.

(A) The number of M. tuberculosis per infected DC at 4, 24, 48, 96 hours after infection in different subcellular compartments. The phagolysosomal mycobacteria are characterized by enclosure of a LAMP-1 labelled membrane and the cytosolic bacteria lack both a membrane and LAMP-1 labelling. Data shown is based on at least 30 cells per time point and is a representative result out of 5 experiments.
(B) The number of live or heat killed M. tuberculosis in macrophages and DCs infected for 96 hours. Amount of mycobacteria determined in LAMP-1 labelled membrane enclosed phagoslysosomes, LAMP-1 lacking membrane enclosed phagolysosomes and in the cytosol. Killed mycobacteria were only present in phagolysosomes while live mycobacteria were translocated to the cytosol.
(C) The number of M. leprae per infected DC at day 4 and 7 in different subcellular compartments. The phagolysosomal mycobacteria are characterized by enclosure of a LAMP-1 labelled membrane, phagosomal bacteria by enclosure of a membrane not labelled for LAMP-1 and the cytosolic bacteria lack both a membrane and LAMP-1 labelling. Data shown is based on at least 30 cells per time point.

### FIG. 4

M. Bovis BCG does not Translocate from the Phagolysosome
(A) The number of M. bovis BCG per infected DC at 2,4 and 7 days in different subcellular compartments. The number of bacteria as determined in LAMP-1 labelled membrane enclosed compartments denoted as phagolysosomes, in phagosome defined as membrane enclosed compartments lacking LAMP-1 and in compartments lacking both membrane and LAMP-1 labelling defined as the cytosol.
(B) The colony forming units (CFU) determined for M. bovis BCG infected DCs. Multiple experiments from which a representative figure is shown, all demonstrated that the CFU increases over time, suggesting that replication occurs.
(C) Representative EM image of DC infected with M. bovis BCG for 7 days and immunogold labelled against LAMP-1. Asterisks indicate phagolysosomal M. bovis BCG, L: lysosomes, M: mitochondria, N: nucleus, ER: endoplasmic reticulum, bar: 200 nm.

### FIG. 5

M. Tuberculosis RD1 Mutants do not Translocate from the Phagolysosome
(A) The number of M. tuberculosis Tn::CFP10 per infected DC at 3 and 7 days in phagolysosomes defined as membrane enclosed LAMP labelled compartments, phagosomes defined as unlabeled membrane enclosed compartments and in the cytosol. This mutant does not translocation to the cytosol and replicates in the phagolysosomes to on average 17 bacteria per infected cell at day 7.
(B) The average number of M. tuberculosis ΔespA, M. tuberculosis ΔespA reconstituted with p3616 and M. tuberculosis Rv per infected DC 7 days after infection. The number of bacteria was determined in LAMP labelled membrane enclosed phagolysosomes, not labelled membrane enclosed phagosomes and in the cytosol. The espA deletion mutant does not translocate while the reconstituted mutant (del espA+p3616) and the wild type M. tuberculosis (Rv) translocate to the cytosol.
(C) Representative EM image of DC infected with M. tuberculosis ΔespA for 7 days; immunogold labelled for LAMP-1 demonstrates that M. tuberculosis ΔespA remains in a membrane enclosed LAMP labelled compartment. Asterisks indicate phagolysosomal M. tuberculosis ΔespA, L: lysosomes, M: mitochondria and bar: 200 nm.

### FIG. 6

Schematic representation of the subcellular pathway of different types of mycobacteria within the host cell. Left panel represents the current view in which mycobacteria reside in an ‘early’ phagosome. The two middle panels show traffic of M. bovis BCG and M. tuberculosis Tn::CFP10 after uptake, both residing and multiplying in a LAMP-1 containing membrane enclosed compartment which fuses with lysosomes. Right panel shows virulent M. tuberculosis present in phagolysosomes and the subsequent translocation to the cytosol. Here multiplication occurs and access to the MHC I pathway is provided.

### Legend Supplementary Figures

Supplementary FIG. 1 MHC I not present on the phagolysosome.

(A) The labelling density (LD) of MHC I on different cellular compartments in DCs infected for 2 hours with M. tuberculosis. The LD was determined as number of gold per μm membrane in the ER, the phagosomal membrane (phago), Golgi complex and plasma membrane (PM) and as a control for the background on mitochondria (mito).
(B,C) Representative electron micrographs of the cells used in (A) demonstrate that the MHC I labelling in the Golgi complex and on the PM and ER (red circles) but on the phagolysosome the labelling is comparable to the background labelling.
Asterisks indicate phagosomal M. tuberculosis, G; Golgi complex, M; mitochondria, MTOC: microtubule-organizing centre, N: nucleus, ER endoplasmic reticulum, bars: 200 nm

### Example 2

Initial host-pathogen encounters include bacterial interactions with epithelial tissues that serve as physical barriers to invasion and infection. Additionally, host phagocytes and antigen presenting cells, such as macrophages and dendritic cells (DCs) have a significant role in innate host resistance to infection and contribute to the generation of adaptive immune responses. These myeloid cells internalize microbes into membrane bound organelles termed phagosomes that mature and fuse with lysosomes. Phagolysosome fusion creates an acidic environment rich in hydrolytic enzymes that degrade and kill bacteria. Moreover, proteolysis of bacteria in these compartments generates antigens that may elicit MHC or CD1 restricted T cell responses.

Intracellular pathogens commonly avoid lysosomal fusion through the manipulation of host signal transduction pathways and alteration of endocytic traffic resulting in privileged replicative niches. In contrast, Listeria monocytogenes and Shigella flexneri lyse the phagosomal membrane and escape from the endocytic system into the host cytosol where they replicate and are able to spread to neighboring cells via actin-based motility (Stevens et al., 2006). Nearly all intracellular pathogens have specialized to manage their fates as “endosomal” or “cytosolic” pathogens. Despite the partial cytosolic localization with low percentages of Mycobacterium marium (Stamm et al., 2003; Stamm et al., 2005) it is currently thought that the most successful pathogenic mycobacterium, M. tuberculosis, persists and replicates within the phagosomes of macrophages. Here it prevents lysosomal fusion and maintains extensive communication with early endosomal traffic in a fashion that is thought to provide access to nutrients for survival and growth. (Orme, 2004; Vergne et al., 2004; Russell et al., 2002; Kang et al., 2005; Russell, 2001; Pizarro-Cerda and Cossart, 2006). In this study we arrive at a different conclusion.

### Results

M. Tuberculosis and M. Leprae Reside in a Phagolysosome Early after Phagocytosis

The subcellular localization of M. tuberculosis and M. leprae was analyzed in freshly isolated human monocyte-derived DCs. DCs were differentiated from human CD14+ monocytes precursors for 5 days in GM-CSF and IL-4, and subsequently infected with M. tuberculosis H37Rv or M. leprae. Samples were fixed at various times after infection (2-48 hours) and processed for cryo-immunogold electron microscopy (Peters et al., 2006). We analyzed the localization of early and late endosomal markers to the M. tuberculosis or M. leprae phagosome. Two hours after infection, the phagosome lacked the early endosomal markers transferrin receptor (TfR) and early endosomal autoantigen 1 (EEA1), which instead were exclusively localized to early endocytic and recycling endosome membranes (Table 1). The phagosome was also negative for the late endosomal cation-independent mannose 6-phosphate receptor (Table 1). In contrast, both M. tuberculosis and M. leprae phagosomal membranes labelled for the lysosomal associated membrane proteins LAMP-1, LAMP-2, CD63 and the major lysosomal aspartic proteinase cathepsin D (FIG. 1A-D and Table 1). In immature DCs, these makers differentially localize in multivesicular and multilamellar lysosomes such as the MHC class II compartment (MIIC) (Peters et al., 1991), with LAMP-1 and LAMP-2 localized on the limiting membrane, CD63 on internal membranes and cathepsin D in the lumen. Following the maturation of DCs, the multivesicular/multilamellar nature of MIICs is modified and all transmembrane proteins (LAMP-1, LAMP-2 and CD63) localize to the limiting membrane of the mature DC lysosome (MDL) (van der Wel et al., 2003). The efficient delivery of these molecules to the phagosome following infection was visualized by the direct fusion of multivesicular lysosomes with the phagosome (FIGS. 1B and B′ arrow heads).

The fusion of lysosomes with the M. tuberculosis phagosome at early time points led us to investigate if LAMP-1 accumulated on phagosomes over time. Over the course between 2 and 48 hours of infection, the average labelling density of LAMP-1 on M. tuberculosis and M. leprae phagosomes remained stable (FIG. 2A) and had levels that were only slightly lower than the lysosomal membranes monitored in the same cells. To determine if the ER contributed to the phagocytosis of either microbe, immunogold labelling was performed on thawed cryo-sections for MHC class I and two ER resident proteins: the cytosolic epitope of MHC class I peptide transporter (TAP) and Protein Disulphide Isomerase (PDI), a soluble ER protein. None of these molecules were detected within or on M. tuberculosis or M. leprae phagosomal membranes at multiple time points (Table 1 and Supplementary FIG. 1). Quantification of the MHC class I labelling density in the ER and on the phagosomal membrane demonstrated that the levels in the phagosome do not rise above background levels of labelling seen in mitochondria (Supplementary FIG. 1). Furthermore, despite the close proximity of ER cisternae to the phagosomal membrane, fusion between the membranes was not observed (n>1000). Thus, following the infection in DCs, the mycobacteria reside in a compartment that readily fuses with lysosomes and forms independent of the ER.

Live M. Tuberculosis and M. Leprae Translocate from the Phagolysosome to the Host Cytosol of Non-Apoptotic Cells

It is thought that in macrophages, the access of the phagosome to the early endocytic system enables M. tuberculosis and M. leprae to evade acidification and degradation, and permits growth by allowing extracellular nutrients to reach replicating bacteria. The localization of almost all M. tuberculosis to a phagolysosomal compartment in DCs during the first two days of infection led us to investigate acidification of the phagosomes. Lysotracker-Red experiments demonstrated that after 20 hours of infection with live M. tuberculosis 24% of the phagosomes were acidified while 87% of phagosomes infected with dead bacteria were acidified at the same time point. These results suggest that in 76% of M. tuberculosis containing phagolysosomes the bacteria are not likely exposed to degradation.

To investigate the intracellular survival and growth in these compartments, DCs were infected with M. tuberculosis and plated in replicate wells of a 24-well plate. At each time point, DCs were lysed and the number of colony forming units (CFU) per well was enumerated. During the initial 48 hours of infection, the titer of M. tuberculosis remained constant indicating no net growth in DC culture over this time (FIG. 2B). Throughout this time period, M. tuberculosis were found exclusively in phagolysosomes, as shown above (FIG. 1).

The slow growth kinetics of M. tuberculosis and the failure of early endocytic vesicles to reach the phagolysosome during the first 48 hours of infection indicate that the phagolysosomal compartment restricts bacterial replication. However, following this period, the titer of M. tuberculosis increased steadily over the next 48 hours of culture (FIG. 2B). In later experiments, similar growth kinetics were observed and the bacterial CFU titer continued to increase between 3 and 7 day post-infection (data not shown). Thus, M. tuberculosis persist during the initial 48 hours infection period in DCs, but are able to replicate significantly only after that time point. The increase in bacterial CFU titer after day 2 suggested that alterations occur to the phagolysosome that create a more favourable growth environment. To investigate the intracellular localization of the bacteria in this timeframe, DCs infected with M. tuberculosis were fixed and processed for immunofluorescence (van der Wel et al., 2005) or cryo immunogold labelling with anti-LAMP-1 and anti-cathepsin D antibodies. After 4 h of infection, M. tuberculosis primarily localized to LAMP-1 and cathepsin D positive phagolysosomes and the amount of bacteria that resided in LAMP-1 or cathepsin D negative compartments was negligible (FIG. 2C). At 48 hours after infection, occasionally, bacteria were found that lacked the characteristic electron lucent zone (Armstrong and Hart, 1971) and did not label for LAMP-1 (FIGS. 3A, A′ and A″). Importantly, these bacteria were not present in membrane-enclosed compartments and were localized to the cytosol. Strikingly, inspection of cells infected for 96 h revealed that the percentage of cytosolic M. tuberculosis increased with a function of time and that larger clusters of bacteria were observed which were not in LAMP-1 or cathepsin D positive compartments (FIGS. 2D and 3B). High magnification images and movies of electron tomographic reconstructions of individual bacteria confirmed that these bacteria lacked phagolysosomal membranes despite residing in close proximity to LAMP-1 or cathepsin D positive lysosomes (FIG. 4 A-D and supplementary FIG. 2). Clusters of M. tuberculosis present in the cytosol are abundant in DCs infected for 4 and 7 days. Of all the -non-apoptotic-infected DCs counted at day 4 and 7 about 32% and 57% respectively had cytosolic mycobacteria. From these results, we conclude that at later stages after infection a large subset of intracellular M. tuberculosis reside in the cytosol of a large proportion of cells. M. leprae infected DCs examined at 4 and 7 days after infection (FIG. 4E and supplementary FIG. 2B) were also found in the cytosol.

To determine if the appearance and large clusters of cytosolic bacteria could be associated with growth of M. tuberculosis, the number of phagolysosomal bacteria and cytosolic bacteria were quantified over time using the absence of LAMP-1 labelling and a phagolysosomal membrane as obligatory features. The number of cytosolic M. tuberculosis per cell rose sharply between 2 and 4 days, increasing approximately 10-fold, while the number of phagolysosomal bacteria increased at a much slower rate (FIG. 4F). Likewise, larger clusters of M. tuberculosis were observed in the cytosol than in phagolysosomes. This progressively increased over time to an average of 13 bacteria in a cluster per cell in 4 days in the cytosol while those numbers remained around 6 in the phagolysosome for the wild-type M. tuberculosis. In no instances did we observe LAMP-1 in the absence of phagosomal membrane, confirming our ability to observe membranes surrounding the bacteria. Similar observations were made in M. tuberculosis infected human monocyte derived macrophages (Supplementary FIG. 3) and THP1 cells (not shown) after 4 days.

To determine if phagolysosomal translocation required an active process of mycobacteria, we examined the localization of heat-killed M. tuberculosis in DCs and macrophages. In both cell types, heat-killed M. tuberculosis resided exclusively in phagolysosomes that were positive for LAMP-1 (FIG. 4G). It is noteworthy that the number of heat-killed bacteria per phagolysosome is comparable to the number of phagosomal bacteria in the live infection, indicating that bacterial burden alone in the phagolysosome is not sufficient for the cytosolic phenotype.

To exclude the possibility that the appearance of cytosolic bacteria was due to reduced viability of infected DCs, we assayed the induction of apoptosis in infected DCs relative to the number of cytosolic mycobacteria. Apoptosis was analyzed using electron microscopy based on morphological features described as hallmarks for apoptosis (Kerr et al., 1972) and by immunofluorescence using Caspase 3 labelling on serial semithin sections on identical samples (van der Wel et al., 2005). Using both techniques, the percentage of apoptotic cells increased slightly between 4 and 96 h after infection, however, a similar increase was observed in control uninfected DCs (data not shown). Furthermore, the percentage of cells containing cytosolic bacteria was 3-4 times greater than the percentage of apoptotic cells (FIG. 4H) showing that the translocation of mycobacteria to the host cytosol occurs in non-apoptotic cells.

Translocation to the Host Cytosol Requires the Mycobacterial Genes CFP-10 of the RD1 Region and espA

Since phagolysosomal translocation required live M. tuberculosis we investigated whether only virulent mycobacteria translocate to the cytosol. To address this, we compared the intracellular localization of the widely used vaccine strain M. bovis BCG and that of virulent M. tuberculosis H37Rv using both fluorescence microscopy and electron microscopy. Strikingly, BCG was restricted to membrane-enclosed compartments positive for LAMP-1 and cathepsin D at 2, 4, and 7 days of infection and no cytosolic mycobacteria were detected in these samples (FIGS. 5A, B). Although BCG failed to enter the cytosol, the number of phagolysosomal BCG and the bacterial titer increased over time (FIG. 5C, D). This result reinforces that translocation to the cytosol does not occur simply by mycobacteria outgrowing its phagolysosomal space.

Dissection of the genetic differences between M. tuberculosis and BCG identified several large deletions from BCG that are present in M. tuberculosis and M. leprae (Harboe et al., 1996; Gordon et al., 1999; Behr et al., 1999; Philipp et al., 1996). From these 16 regions of difference (RD1-16) only RD1 is absent from all BCG strains thus far tested (Mostowy et al., 2002; Tekaia et al., 1999; Brosch et al., 2002). RD1 is part of a 15-gene locus known as ESX-1 that encodes a specialized secretion system dedicated to the secretion of CFP-10 and ESAT-6. In addition to the genes encoded in ESX-1, a second unlinked locus encoding espA is required for CFP-10 and ESAT-6 secretion (Fortune et al., 2005). The deletion of RD1 in BCG and the importance of the ESX-1 secretion system in virulence (Brodin et al., 2006) led us to test whether CFP-10 and ESAT-6 were required for M. tuberculosis translocation to the cytosol. This was first examined by using a M. tuberculosis strain containing a transposon insertion in cfp-10 (Rv3874), which prevents the synthesis of CFP-10 and ESAT-6 (Guinn et al., 2004). Like BCG, this mutant failed to enter the host cytosol over the course of 7 days of infection and resided in LAMP-1 positive compartments (FIG. 6A). Next, we used an ΔespA strain of M. tuberculosis to determine if the secretion of CFP-10 and ESAT-6 is required for the cytosolic phenotype. Following infection of DCs, the ΔespA strain and the ΔespA strain carrying the empty complementing vector espA pJEB; not shown) localized to LAMP-1 positive phagolysomes and a low percentage mycobacteria were detected in host cytosol (FIG. 6B, C). Strikingly, complementation of espA restored the number of cytosolic bacteria to a similar level as wild-type M. tuberculosis (FIGS. 6B, D), demonstrating a role for the ESX-1 system and the secretion of CFP-10 and ESAT-6 in the translocation of M. tuberculosis from the host endocytic system.

To determine in an independent approach if M. tuberculosis replicates in the cytosol and the Tn::CPF-10 mutant in the phagolysomes, we determined the amount of FtsZ, a bacterial tubulin like protein. FtsZ is critical for the cell division process in many prokaryotes including mycobacteria and is transiently higher expressed during cytokinesis (Margolin, 2005). The relative immunogold labelling index for FtsZ was determined on mycobacteria in the cytosol and in phagolysosomal compartments at different times of infection and compared to the labelling on cellular compartments as control (supplementary FIG. 4). The data demonstrate at 7 days of infection the highest amount of FtsZ in cytosolic M. tuberculosis relative to phagolysosomal bacteria suggesting that M. tuberculosis preferably replicates in the cytosol. In contrast, the Tn::CFP-10 mutant replicates in the phagolysosomal compartments.

### Translocation to the Host Cytosol is Followed by Cell Death

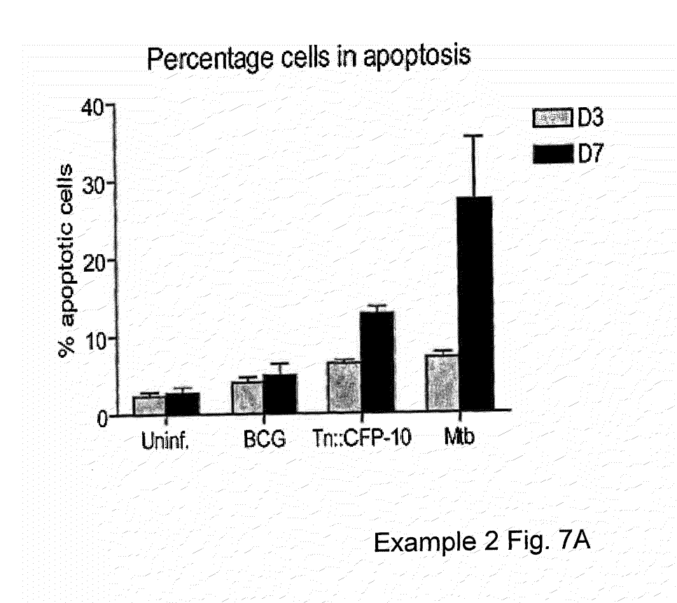

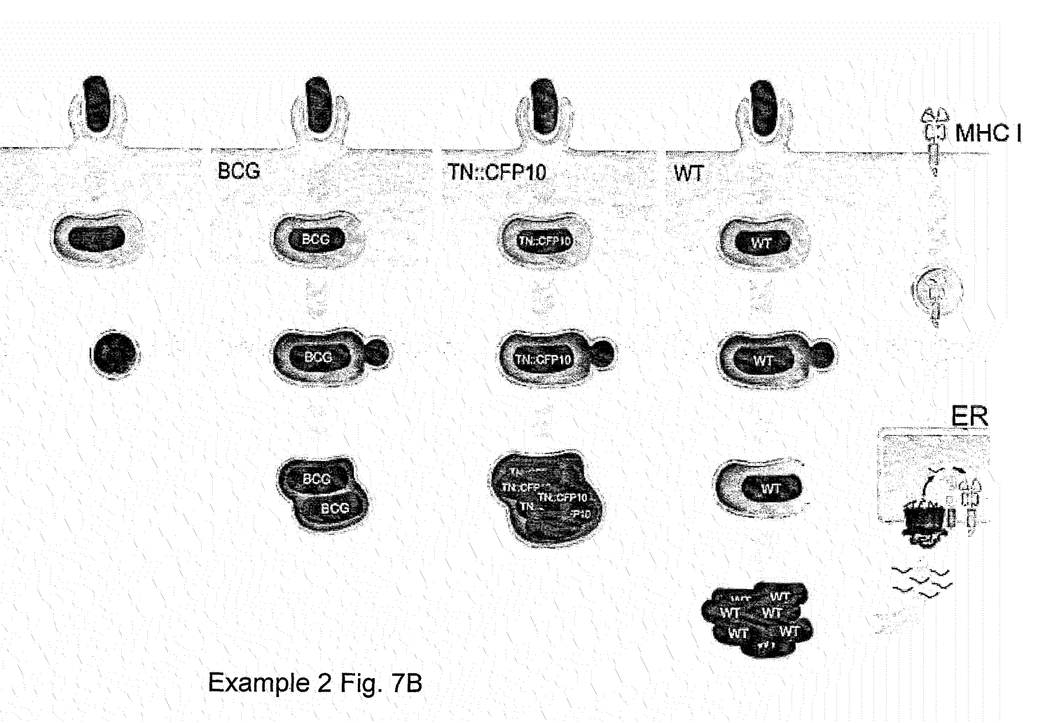

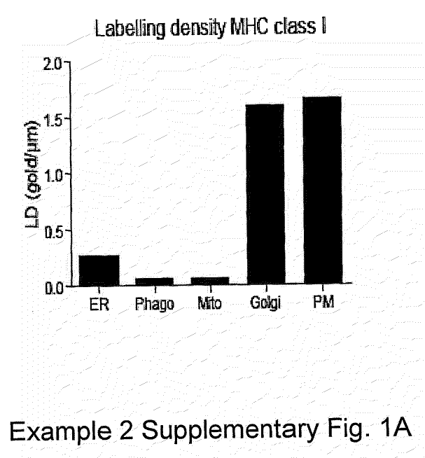

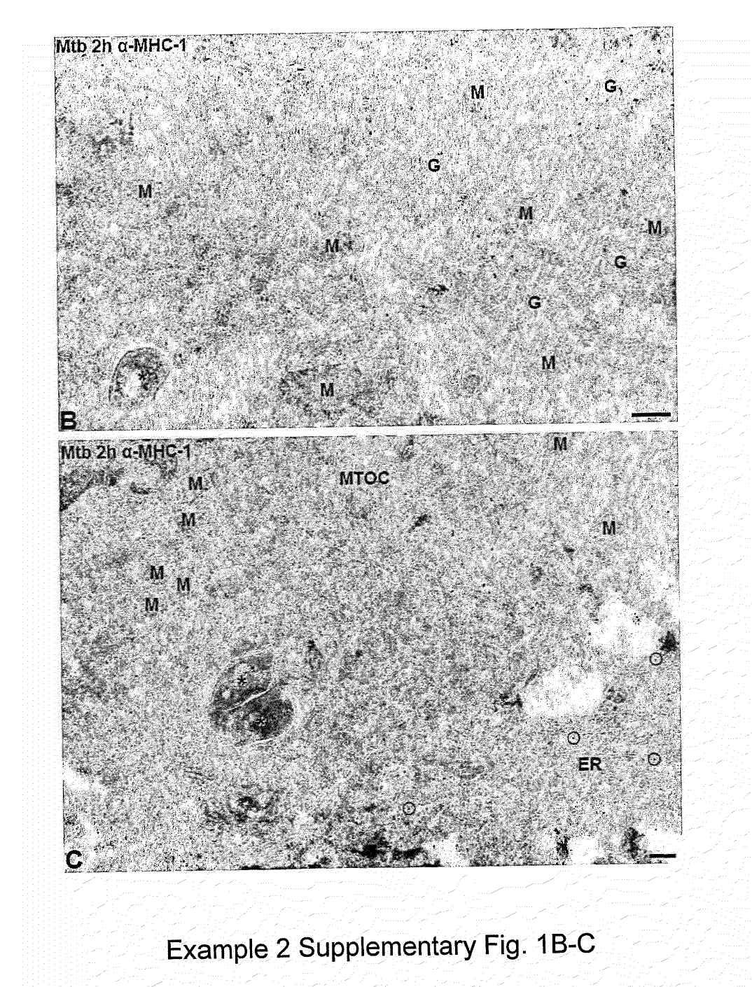

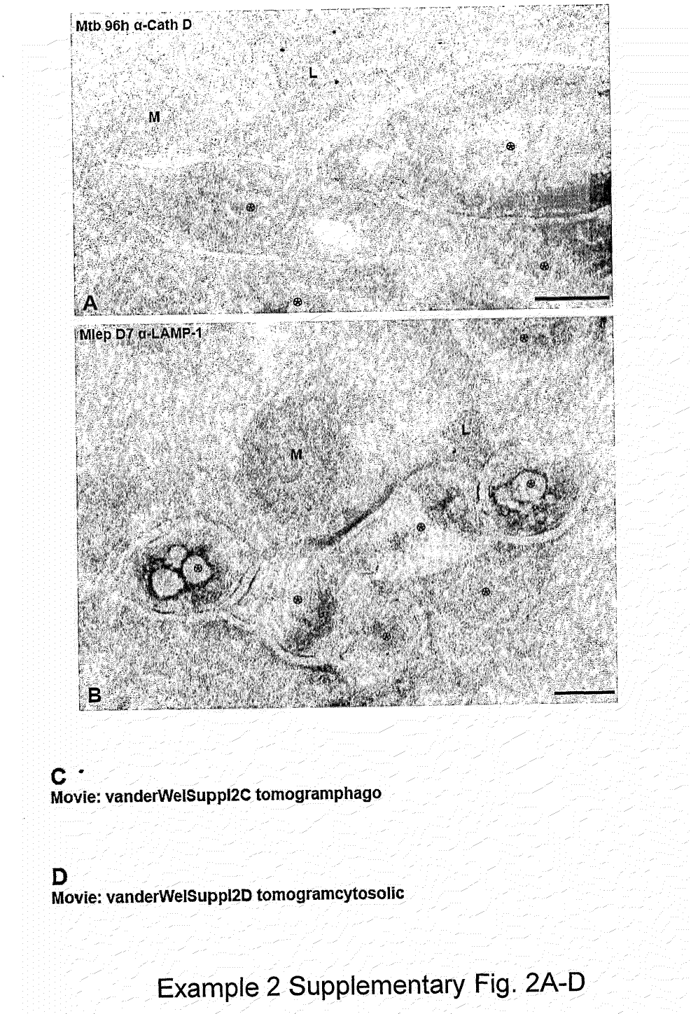

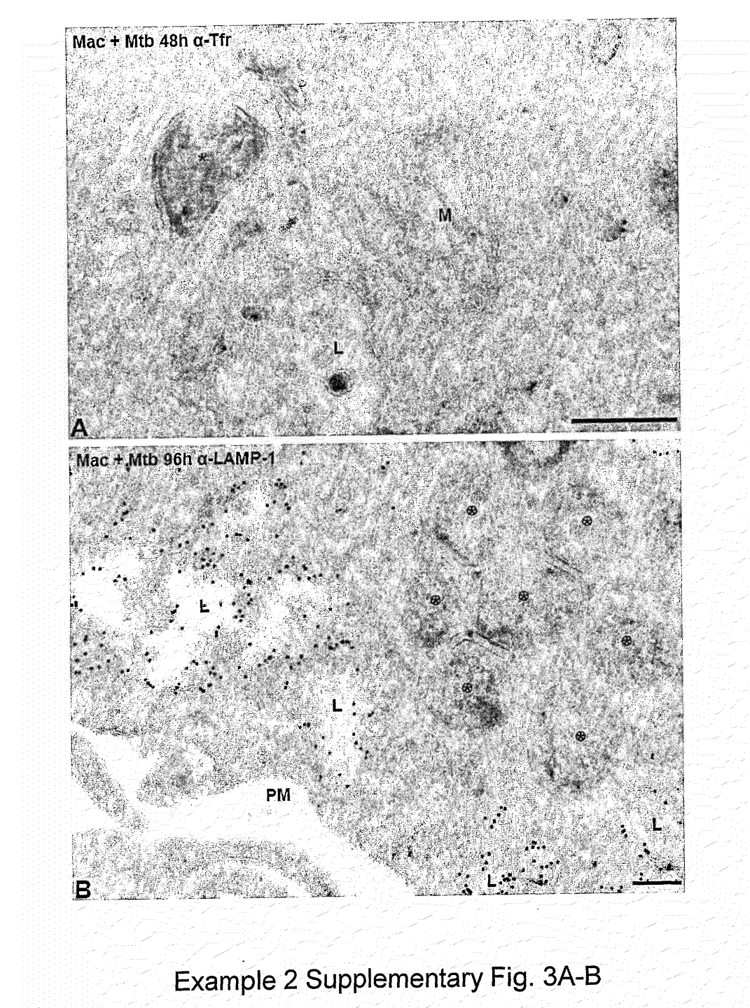

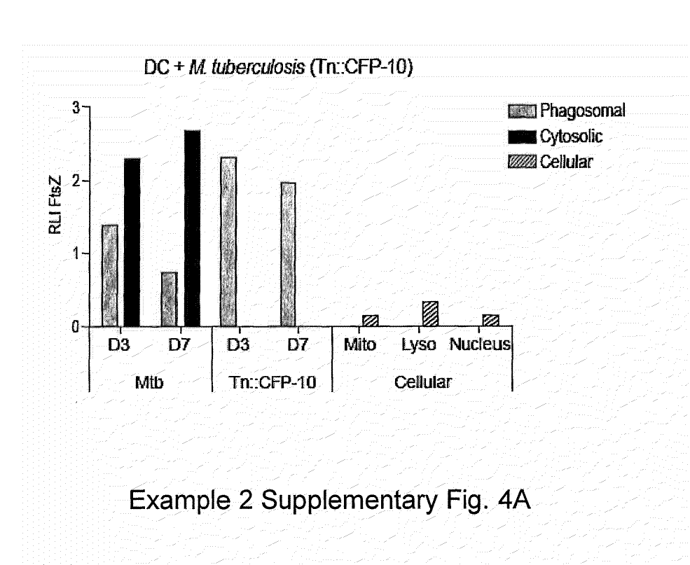

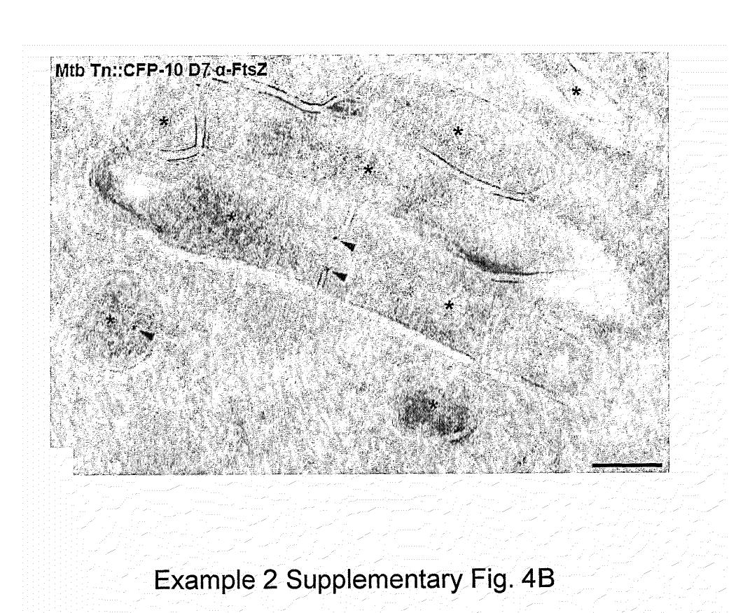

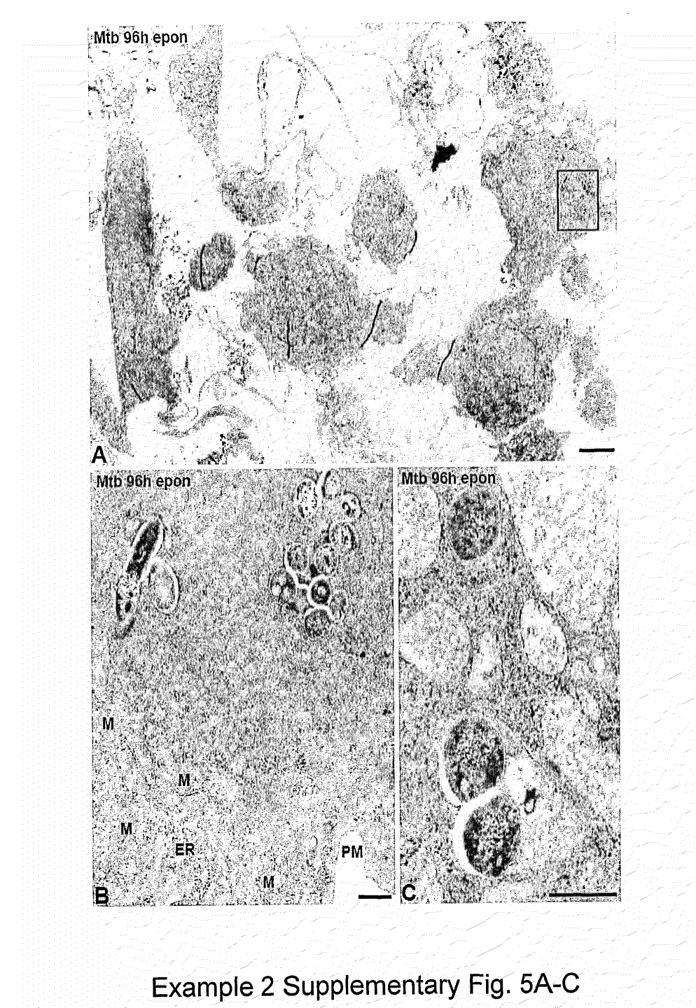

TAB Others have demonstrated that M. tuberculosis and more specifically ESAT-6 can induce apoptosis (Placido et al., 1997; Keane et al., 1997; Riendeau and Kornfeld, 2003; Lee et al., 2006; Derrick and Morris, 2007). We observe in DCs cultures, infected with M. tuberculosis for 7 days that the amount of cell death based on Caspase 3 and EM is significantly increased. Interestingly DCs infected with mutant M. tuberculosis Tn::CFP-10 showed a lower amount of Caspase 3 positive apoptotic cells (FIG. 7A). Importantly, the translocation of M. tuberculosis to the cytosol precedes the induction of apoptosis (see also FIG. 4H).

### Discussion

Previous studies showed some evidence for M. tuberculosis that appeared to be free in the cytoplasm; however in the absence of mechanism (Myrvik et al., 1984; Leake et al., 1984; McDonough et al., 1993) using traditional ‘plastic embedded’ electron microscopy. It has been difficult to confirm these results as this technique does not allow immunogold labelling and does not visualize distinctly the host phagolysosome and mycobacterial membrane bilayer (see Supplementary FIG. 5 and compare with for example FIG. 1B and the electron tomographic reconstruction in FIG. 4 and moves in supplementary FIGS. 2C,D). The prevailing paradigm has remained that M. tuberculosis reside in the endocytic system (Orme, 2004; Vergne et al., 2004; Russell et al., 2002; Kang et al., 2005; Russell, 2001; Pizarro-Cerda and Cossart, 2006). Mycobacterium localization in infected macrophages has been extensively studied for over 40 years using an array of techniques and a number of Mycobacterium species as model organisms for M. tuberculosis. In general, the majority of these experimental systems only focused on the first 48 hours following infection and were often performed with avirulent mycobacteria. Here we have used an extended time course to examine the localization of M. tuberculosis and M. leprae for up to 7 days of infection. In our assays, the excellent preservation of cellular membranes in cryosections, coupled with immunological detection of endocytic markers allowed the quantitative assessment of mycobacterial localization to the cytosol only at times beyond 2 days of infection.

In addition to M. tuberculosis, the RD1 locus is also present in M. bovis, M. kansasii, M. marinum, M. africanum, and M. leprae (Berthet et al., 1998; Harboe et al., 1996). The ESX-1 region has an important role in the virulence of M. tuberculosis (Lewis et al., 2003; Hsu et al., 2003; Stanley et al., 2003). The genes encoded in the ESX-1 region are predicted to form a specialized secretory apparatus that secretes CFP-10 and ESAT-6. Pathogens such as L. monocytogenes that lyse host phagosomes and replicate in the host cytosol induce potent CD8+ T cell responses (Glomski et al., 2002; Schuerch et al., 2005). Along these lines it is interesting to speculate that an analogous mechanism may function during M. tuberculosis infection. The intracellular expression patterns of CFP-10, ESAT-6 and EspA have not been characterized in detail, however, they are clearly expressed following infection of human macrophages. Guinn et al. have reported that M. tuberculosis lyses host cells and spreads to uninfected macrophages over a 7 day time course, and that this occurs in a RD1-dependent manner (Guinn et al., 2004). Recently, M. marinum has been shown to escape with low relative numbers from phagosomes in infected macrophages and spread to neighbouring cells via actin based motility (Stamm et al., 2003; Stamm et al., 2005). These processes also involve CFP-10 and ESAT-6 (Gao et al., 2006). In contrast we did not find any evidence for actin tails for M. tuberculosis.

The immune response to M. tuberculosis is a dynamic process involving both CD4+ and CD8+ T cells (Flynn and Chan, 2001), which predominate as the major INFγ secreting cells at different stages of infection: CD4+ T cells dominate during acute infection and CD8+ T cells during persistent infection (Lazarevic et al., 2005). How antigens from intracellular bacteria gain access to the MHC class I antigen loading pathway in the ER remains an intense area of study. Several groups have suggested direct fusion between the ER and phagosome during phagocytosis (Houde et al., 2003; Ackerman et al., 2003; Guermonprez et al., 2003), however, quantitative assessment of ER markers on both model latex bead phagosomes and M. avium containing phagosomes contradict those findings (Touret et al., 2005). Similarly, we find no evidence for the localization of ER markers with a cytosolic epitope to the mycobacteria containing phagosome after infection, but rather we suggest that M. tuberculosis and M. leprae antigens presented by MHC class I are most likely derived from bacteria that have entered the host cytosol as shown here (see FIG. 7B). Recent in vivo work (Majlessi et al., 2005) and unpublished data presented at the TB Keystone meeting 2007 confirm this suggestion by showing a significant increase of MHC class I restricted CD8+ T cell response in a recombinant BCG strain in which the extended RD1 region is introduced (R. Billeskov and J. Dietrich, personal communication) or by showing that the T cell response to CFP-10 and ESAT-6 is eliminated in M. tuberculosis mutations affecting the function of the ESX-1 secretion system (S. Behar, personal communication)

It is significant that BCG, which is used in many countries worldwide as a mycobacterial vaccine strain remains restricted to the phagolysosome following infection of DCs and macrophages, whereas virulent M. tuberculosis does not (FIG. 7B). BCG vaccination has questionable efficacy against the highly infectious pulmonary form of tuberculosis, and it fails to generate a strong MHC class I restricted T cell response. The work presented here emphasizes that avirulent BCG fail to translocate the phagolysosome and suggests this may account for their poor capacity to stimulate critical CD8+ T cell responses through MHC class I (FIG. 7B). Interestingly, innovative vaccine approaches have genetically engineered BCG to express LLO as a mechanism to generate more potent MHC class I-restricted responses. Indeed, LLO+BCG are more effective vaccines than the isogenic BCG parental strain (Grode et al., 2005). Designing vaccines that mimic virulent strains in translocating into the cytosol is likely to be a critical step forward in producing more effective vaccines for tuberculosis.

### Material and Methods

### Human Cell Cultures

Peripheral blood mononuclear cells (PBMC) were isolated from healthy human donors as previously described (Porcelli et al., 1992). CD14+ monocytes were positively selected from PBMC using CD 14 microbeads and magnetic cell separation (Miltenyi Biotec, Auburn Calif.). Immature human monocyte-derived DCs were prepared from CD14+ monocytes by culture in 300 U/ml of granulocyte-macrophage colony-stimulating factor (GM-CSF, Sargramostim, Immunex, Seattle, Wash.) and 200 U/ml of IL-4 (PeproTech, Rocky Hill, N.J.) for 5 days in complete RPMI medium (10% heat-inactivated FCS/20 mM Hepes/2 mM L-glutamine/1 mM sodium pyruvate/55 μM 2-mercaptoethanol/Essential and non-essential amino acids). GM-CSF and IL-4 were replenished on day 2, day 5, and day 9 after isolation. Macrophages were prepared by culture of CD14+ monocytes in IMDM with 10% human AB serum, 2 mM L-glutamine, and 50 ng/mL M-CSF (PeproTech, Rocky Hill, N.J.).

### Mycobacterial Infections

M. tuberculosis strains and Bacillus of Calmette and Guérin (BCG) were grown to mid-logarithmic phase from frozen stocks in 7H9 Middlebrook media containing OADC enrichment solution and 0.05% Tween-20 for 1 week at 37° C. The wild-type M. tuberculosis strain used in these studies was H37Rv expressing green fluorescent protein (GFP) (Ramakrishnan et al., 2000). The BCG strain was provided by Barry Bloom. The Tn::Rv3874 (cfp-10) and the ΔespA strain (delta3616) have been previously described (Guinn et al., 2004; Fortune et al., 2005). The ΔespA strain complemented strain encodes espA under the control of its native promoter on an integrating vector (delta3616+p3616). The construct has been shown to complement the ΔespA mutation for ESAT-6 secretion (S. Fortune, Personal communication). The delta3616 pJEB—the espA deletion strain with the empty vector. M. leprae were purified from mouse footpads as previously described and used in experiments one day after isolation (Adams et al., 2002).

For in vitro infections, bacteria were harvested and suspended in RPMI containing 10% FCS, 2% human serum and 0.05% Tween 80, followed by washing in RPMI complete media. Cultures were filtered though a 5 μM syringe filter to obtain cell suspensions and counted using a Petroff-Houser chamber. Bacteria were added to DCs and macrophage cultures at an MOI˜10 and plates were centrifuged for 2 min at 700 rpm prior to incubation at 37° C. with 5% CO₂. After 1 h, infected macrophage cultures were washed three times with warm culture media to remove free mycobacteria. For DC cultures, media was removed after 4 hours of infection, diluted ˜1:6 in prewarmed RPMI complete media, centrifuged at 1000 rpm for 2 min, and resuspended in RPMI complete media supplemented with GMCSF/IL4. Culture wells were washed with RPMI three times to remove any remaining extracellular bacteria prior to replating DCs.

Colony forming units (CFU) were enumerated by lysing infected DCs in sterile water with 0.1% saponin for 5 min. Lysed cells were repeatedly mixed and dilutions were made in sterile saline containing Tween-20. Diluted samples were plated on 7H11 Middlebrook agar plates (Remel) and colonies enumerated after 2-3 weeks of growth.

### Electron Microscopy

Fixed cells were collected, embedded in gelatine and cryosectioned with a Leica FCS and immuno labelled as described previously (Peters et al., 2006). Samples were trimmed using a diamond Cryotrim 90 knife at −100° C. (Diatome, Switzerland) and ultrathin sections of 50 nm were cut at −120° C. using an Cryoimmuno knife (Diatome, Switserland). More details on immunolabeling are in the Supplement.

### Supplementary Material and Methods

Lysotracker-Red staining of Infected DCs.

DCs infected for 20 h with M. tuberculosis H37Rv-gfp or heat-killed M. tuberculosis were plated on fibronectin (20 μg/mL; Sigma) coated glass coverslips. Lysotracker Red D-99 in (200 nM, Molecular Probes) was added for the final 30′ of incubation and cells were fixed with 2% formaldehyde at room temperature. To visualize heat-killed bacteria, coverslips were stained with rabbit anti-LAM antisera (Daniel Clemens, UCLA) followed by donkey anti-rabbit Alexa488-conjugated (Molecular Probes) antibodies. Confocal microscopy images were acquired on a Nikon C-1 confocal microscope with software EZ C1 and the percentage of phagosomes that colocalized with Lysotracker-Red was calculated from images examined in Adobe Photoshop CS v8.0.

### Electron Microscopy

At each time point of the infection, cells were fixed by adding an equal volume of 2× fixative (0.2M PHEM buffer containing 4% paraformaldehyde or 0.4% Gluteraldehyde and 4% paraformaldehyde) to plates immediately after removal from the incubator. Cells were fixed for 24 hours at room temperature and recovered using a cell scraper. Fixed cells were stored in 0.1M PHEM buffer and 0.5% paraformaldehyde until analysis. Fixed cells were collected, embedded in gelatine and cryosectioned with a Leica FCS and immuno labelled as described previously (Peters et al., 2006). Samples were trimmed using a diamond Cryotrim 90 knife at −100° C. (Diatome, Switzerland) and ultrathin sections of 50 nm were cut at −120° C. using a Cryoimmuno knife (Diatome, Switserland). Immunogold labelling was performed using lysosome associated membrane protein 1 and 2 (LAMP-1 and LAMP-2 clone H4A3 and H4B4 from BD Biosciences), cathepsin D (clone 1C11 Zymed), CD63 (M544 Sanquin the Netherlands), mannose 6 phosphate receptor (M6PR a gift from Dr. V. Hsu), EEA1 (Transduction labs Lexington, Ky.), Transferrin receptor (TfR H68.4 (CD71) Zymed), MHC class I (HC10 a gift from Dr. J. Neefjes), FtsZ (a gift from Dr. Rajagopalan), TAP (198.3 a gift from Dr. J. Neefjes) and PDI (a gift from Dr. H. Ploegh). Antibodies were labelled with rabbit anti-mouse bridging serum (DAKO) and protein-A conjugated to 10 nm gold (EM laboratory, Utrecht University). Sections were examined using a FEI Tecnai 12 transmission electron microscope. Quantitation was done according to routine stereological methods. The labelling density and relative labelling index determined for respectively MHC class I and FtsZ was calculated according to Mayhew (Mayhew et al., 2002)

All specimens used for tomography were paraformaldehyde fixed and processed as described.

### Fluorescence Microscopy

Samples were prepared as previously described (van der Wel et al., 2005). The immuno-fluorescence labelling was performed using LAMP-1 and LAMP-2 (clone H4A3 and H4B4 from BD Biosciences), cathepsin D (clone 1C11 Zymed) and Caspase 3 (Asp175 Cell Signaling) antibodies and Texas Red secondary antibody (Mol Probes). The bacteria were stained with cell wall protein (C188 a gift form Dr Brennan Colorado State) and Alexa488 (Mol Probes). Slides were mounted with Vecta-shield media, containing 4,6-diamino-2-phenylindole (DAPI) for nuclear staining (Vector Laboratories, Burlingame, Calif.).

### Brief Description of the Drawings of Example 2

### FIG. 1. In Early Stages of Infection, M. Tuberculosis and M. Leprae Reside in LAMP-1 and Cathepsin D Containing Phagolysosomes.

### (A) LAMP-1 Labelling on Phagosomal Membrane Early in Infection

Immunogold labelling of LAMP-1 on a DC infected with M. tuberculosis for 2 hours on phagolysosomes and lysosomes. For comparison there is no background labelling on the mitochondrium in the same cell. Note that only membranes, perpendicular present in section direction, can be properly stained and thus visualized in cryosectiones as these are negatively stained by Uranyl acetaat. Therefore, membranes appear as electron lucent structures surrounded by an electron dense substrate.

(B) Fusion of Lysosomes with CD63 Labelled Phagosomal Membrane

CD63 labelling on the limiting membrane of the phagolysosome in a DC infected with M. tuberculosis for 2 hours. In addition to labelling several fusion events of lysosomes with the phagolysosome are visible (arrowheads). Note the electron lucent zone between the phagosomal membrane and the electron lucent bacterial cell wall.

(B′) Enlargement of (B) showing fusion event between the limiting membrane of a (multi-vesicular) lysosome and the phagolysosomal membrane.

### (C) Cathepsin D Present in the Phagosomes Early in Infection

DC infected with M. tuberculosis for 2 hours and immunogold labelled for cathepsin D. Label is present in lysosomes and in the phagolysosome.

### (D) M. Leprae Localized in LAMP-1 Labelled Phagosome

Labelling of LAMP-1 on phagolysosome of DC infected with M. leprae for 48 hours.

Asterisks indicate mycobacteria in phagolysosomes, M: mitochondrium, L: lysosome, arrowheads: fusion profiles. All images are from cryo-immunogold labelled cryosections. Bar: A) 250 nm, B) 200 nm, C) 400 nm and D) 300 nm.

FIG. 2. The Relative Amount of M. Tuberculosis in DCs Increases After 48 Hours of Infection, which Coincides with a Substantial Translocation from the Phagolysosome to the Cytosol.

### (A) LAMP-1 Labelling Density on Phagosomes and Lysosomes

LAMP-1 labelling density (LD): number of gold particles per μm phagosomal membrane as determined on at least 30 phagolysosomes in DCs infected with M. tuberculosis for 2, 24 and 48 hours, and M. leprae 48 hours remains equal and compared to the LD on the limiting membrane of lysosomes (L) slightly lower. For comparison the background labelling on the mitochondria (M) in the same cells is negligible. Error bars represent standard error.

(B) Replication M. Tuberculosis Increases after 48 Hours of Infection in DCs

The colony forming units (CFU) determined for M. tuberculosis infected DCs. Multiple experiments from which a representative figure is shown, all demonstrated that the CFU increased after 48 hours, suggesting that replication was significantly (small error bars, representing standard error) initiated after 48 hours of infection.

(C) M. Tuberculosis Co-Localizes with LAMP-1 and Cathepsin D after 4 Hours

Fluorescence image of DCs infected with M. tuberculosis (green) for 4 hours labelled with anti cathepsin D (red) or LAMP-1 (red) and DAPI (blue) demonstrates that at early stages the bacteria are present in a phagolysosomal compartment. Merged images on the right panel.

(D) No co-localisation of M. tuberculosis with LAMP-1 and cathepsin D after 96 hours Fluorescence images of DCs infected for 96 hours in which large clusters of M. tuberculosis (green) bacteria are present. Most of these clusters do not co-localize with the lysosomal markers cathepsin D (red) and LAMP-1 (red) although individual bacteria were shown (arrow head) to co-localise. Merged images on the right panel.
FIG. 3. Translocation from the Phagolysosome to the Cytosol at High Resolution

### (A) Phagolysosomal and Cytosolic M. Tuberculosis in a DC

Electron micrograph of a DC infected with M. tuberculosis for 48 hours showing different subcellular locations: 1) mycobacteria observed in membrane-enclosed phagolysosomes (asterisk) which are characterized by an electron lucent zone between the phagosomal membrane and the bacterial cell wall and immunogold labelling with LAMP-1 on the phagolysosomal membrane. 2) mycobacteria detected in the cytosol (encircled asterisk) lacking the enclosure of a membrane and the LAMP-1 labelling (more examples in FIGS.: 3B, 6D and supplemental FIGS. 2 and 3B). Not in this image, but detectable in low amounts are mycobacteria in membrane-enclosed compartments lacking LAMP-1, here defined as phagosomal.

(A′) Enlargement of (A) to demonstrate that enlargement of the EM figure allows the identification of the distinguishable layers present in and around cytosolic M. tuberculosis. a) cytoplasm M. tuberculosis, b) plasma membrane of M. tuberculosis which can be discontinuous by the fixation or freezing artefacts, c) lipid rich cell wall also referred to as capsid, f) host cytosol. (A″) Enlargement of (A) indicating additional layers present around phagosomal M. tuberculosis. Layers in the bacteria are identical to the cytosolic layers with the addition of two cellular layers: d) phagosomal or electron lucent space, which varies in size, e) phagosomal membrane, immunogold labelled for LAMP-1.

(B) Large Clusters of Cytosolic M. Tuberculosis after 96 Hours of Infection

Clusters of M. tuberculosis present in the cytosol are abundant in non-apoptotic DCs infected for 96 hours.

(B′) Enlargement of boxed area demonstrating that phagosomal membranes do not surround these bacteria even though the lysosomal membranes are well distinguished and labelled with LAMP-1.

L: lysosomes, M: mitochondrium, asterisk: mycobacteria in phagolysosomes,

encircled asterisks: cytosolic mycobacteria. All images are from cryo-immunogold labelled cryo-sections. Bar: A) 300 nm, B) 500 nm.

### FIG. 4. Tomograms of Cryosections and Number of Live M. Tuberculosis Increases in the Cytosol

### (A) Tomogram of M. Tuberculosis in Phagolysosome

A 5 nm thick tomographic slice from a 60 nm cryosection that shows a DC infected with M. tuberculosis for 48 hours, immuno-labelled for LAMP-1 with 10 nm gold particles. The reconstruction was made from a −60 degree to +60 degree tilt series taken in 1 degree increments. The reconstruction was made using weighted back projection using the IMOD software (Kremer et al., 1996). Movie available in Supplementary FIG. 2C.

Asterisk: mycobacteria in phagolysosomes, N: nucleus, M: mitochondrium, G: golgi.

### B) Model of the Phagolysosomal M. Tuberculosis Tomogram

A coarse IMOD model of the tomogram in (A). The inner side of the mycobacterial (Mtb) cell wall was used to draw the model of the bacteria (red) and the total phagosomal (Ph) and nuclear envelope (NE) membrane was used to draw the model of the cellular membranes (yellow).

### (C) Tomogram of M. Tuberculosis in Cytosol

A 5 nm thick tomographic slice from a 200 nm thick cryosection of DCs infected with M. tuberculosis for 96 hours immuno-labelled for LAMP-1 with 10 nm gold particles. The reconstruction was made from a −60 degree to +60 degree tilt series taken in 1 degree increments. The reconstruction was made using weighted back projection using the IMOD software. The specimens were sectioned in thick (200 nm) sections to enlarge the chance of including membranous structures however; no membranes surrounding the bacteria were detected. Movie available in Supplementary FIG. 2D. Encircled asterisk: cytosolic M. tuberculosis, M: mitochondrium, L: lysosome.

### (D) Model of the Cytosolic M. Tuberculosis Tomogram

IMOD model based on tomogram from (C). The inner side of the mycobacterial (Mtb) cell wall was used to draw the model of the bacteria (red) and the lysosomal (L) membrane was used to draw the model of the lysosomes (yellow).

### (E) Quantification of Number of M. Leprae In Different Subcellular Compartments

The number of M. leprae per infected DC as observed on immunogold EM labelled cryo-sections at day 4 and 7 in phagolysosomes, phagosomes and in the cytosol. The phagolysosomal, phagosomes and cytosolic mycobacteria are characterised as described in FIG. 3A. Error bars represent standard errors. M. leprae resides in all compartments.

### (F) Quantification of Increased Replication of M. Tuberculosis in Cytosol

The number of M. tuberculosis per infected DC at 4, 24, 48 and 96 hours after infection in different subcellular compartments as observed on immunogold EM labelled cryo-sections. Data are based on at least 30 cells per time point and is a representative result out of 5 independent experiments. Error bars represent standard errors

### (G) Live not Dead M. Tuberculosis Translocates in Cytosol of Both DCs and Macs

The number of live or heat-killed M. tuberculosis per macrophage and DC infected for 96 hours in phagoslysosomes and in the cytosol. Error bars represent standard error. Killed mycobacteria were only present in phagolysosomes while live mycobacteria were translocated to the cytosol.

### (H) Translocation to Cytosol Precedes Induction of Apoptosis

Percentage cells containing cytosolic bacteria (Cytosolic) or showing apoptotic features based on the morphology in ultrathin cryosections visualised with the electron microscope (Apoptotic EM) or the presence of Caspase 3 with fluorescence microscopy (Apoptotic Casp3) at different time points after infection. After 96 hours the percentage of cells with cytosolic bacteria rapidly increases until 22% while the percentage apoptotic cells remains below 7%.

FIG. 5. M. Bovis BCG does not Translocate from the Phagolysosome

### (A) Late in Infection M. Bovis BCG Remains Localised in a Lysosomal Compartment

DCs infected with M. bovis BCG (green) for 7 days show co-localisation with cathepsin D or LAMP-1 (red) demonstrating that the bacteria reside in the phagolysosome (see for contrast with M. tuberculosis FIG. 2D).

### (B) M. Bovis BCG Localized in a Membrane Enclosed, LAMP-1 Labelled Compartment

Representative EM image of DC infected with M. bovis BCG for 3 days and immunogold labelled for LAMP-1. M. bovis BCG is contained in phagolysosomes. Asterisks indicate LAMP-1 positive phagolysosomal M. bovis BCG. L: lysosomes, M: mitochondrium. Bar: 200 nm.

(B′) Enlargement of boxed area demonstrating the immunogold labelled phagosomal membrane surrounding the mycobacterial cell wall.

### (C) Replication of M. Bovis BCG in the Phagolysosome

The number of M. bovis BCG per infected DC at 2, 4 and 7 days as observed on immunogold EM labelled cryo-sections in different subcellular compartments as described in FIG. 3A. Error bars represent standard error.

(D) Early Replication of M. bovis BCG

The colony forming units (CFU) determined for M. bovis BCG infected DCs. Multiple experiments from which a representative figure is shown and all demonstrated that the CFU increases over time, suggesting that replication occurs. Error bars represent standard error.

FIG. 6. M. Tuberculosis RD1 Mutants do not Translocate from the Phagolysosome
(A) CFP-10 Mutant of M. tuberculosis Replicates in Phagolysosome

The number of M. tuberculosis Tn::CFP-10 per infected DC at 3 and 7 days as observed on immunogold EM labelled cryo-sections in phagolysosomes, phagosomes and in the cytosol as defined in legend FIG. 3A. This mutant does not translocate to the cytosol and replicates in the phagolysosomes to on average of 17 bacteria per infected cell at day 7. Error bars represent standard error.

(B) ΔespA Mutant M. Tuberculosis Localises in Phagolysosome

The average number of M. tuberculosis ΔespA (delta3616), M. tuberculosis ΔespA reconstituted with espA (delta3616+p3616) and M. tuberculosis H37Rv per infected DC 7 days after infection. The number of bacteria was determined as described for FIG. 3A. The espA deletion mutant does not translocate while the complemented espA mutant (deta3616+p3616) and the wild type M. tuberculosis H37Rv (Mtb) translocate to the cytosol.

(C) ΔespA Mutant M. Tuberculosis Localises in Membrane Enclosed Phagolysosome

Representative EM image of DC infected with M. tuberculosis ΔespA for 7 days immunogold labelled for LAMP-1 demonstrates that M. tuberculosis ΔespA remains in a membrane-enclosed LAMP-1 labelled compartment.

(D) ΔespA Mutant Complemented with espA M. Tuberculosis Localises in Cytosol

Representative EM image of DC infected with M. tuberculosis ΔespA complemented with espA (deta3616+p3616) for 7 days showing cytosolic location; lysosomes and mitochondria show clear membranes.

Asterisks (FIG. 6C) indicate phagolysosomal M. tuberculosis ΔespA, encircled asterisks (FIG. 6D) indicate cytosolic M. tuberculosis ΔespA complemented with espA, L: lysosomes, M: mitochondria. Bar: C) 200 nm and D) 300 nm.

### FIG. 7 Cytosolic M. Tuberculosis Induces Apoptosis and Schematic Representation Subcellular Pathway

### (A) Cytosolic M. Tuberculosis Induces Apoptosis

Percentage apoptotic cells after infection with M. tuberculosis, M. bovis BCG or M. tuberculosis Tn::CFP-10 per infected DC and uninfected control cells at 3 and 7 days as determined with Caspase 3 labelling with fluorescence microscopy. The percentage apoptotic cells rapidly increases after 3 days when DCs are infected with M. tuberculosis while the percentage apoptotic cells remains below 5% for M. bovis BCG and uninfected control cells. M. tuberculosis Tn::CFP-10 infected cells demonstrate an intermediate percentage of apoptosis.

### (B) Schematic Representation of the Subcellular Pathway of Different Types of Mycobacteria

The subcellular pathway of different types of mycobacteria within the host cell. Left panel represents the current view in which mycobacteria reside in an ‘early’ phagosome. The two middle panels show traffic of M. bovis BCG and M. tuberculosis Tn::CFP-10 after uptake, both residing and multiplying in a LAMP-1 containing membrane-enclosed compartment which fuses with lysosomes. Right panel shows virulent M. tuberculosis or M. leprae present in phagolysosomes and the subsequent translocation to the cytosol. Here possible replication, degradation and peptide delivery to the MHC I pathway occurs.

### Legend Supplementary Figures

### Supplementary FIG. 1.

### MHC Class 1 Molecules are not Present on the Phagolysosome.

### (A) Low Labelling Density of MHC Class I on Phagosomes

The labelling density (LD) of MHC class I on different cellular compartments in DCs infected for 2 hours with M. tuberculosis. The LD was determined as number of gold per μm membrane in the ER, the phagosomal membrane (phago), Golgi complex and plasma membrane (PM) and as a control for the background on mitochondria (mito).

(B,C) Ample MHC class I labelling on Golgi and ER but no on phagosome Representative electron micrographs of the cells used in (A) demonstrate the MHC class I labelling in the Golgi complex, on the plasma membrane and ER (red circles). On the phagolysosome no gold labelling is seen.

Asterisks indicate phagosomal M. tuberculosis, G: Golgi complex, M: mitochondrium, MTOC: microtubule-organizing centre, N: nucleus, ER endoplasmic reticulum, bars: 200 nm

### Supplementary FIG. 2.

No Lysosomal Markers Present of Cytosolic Bacteria 4-7 Days after Infection
(A) Late in Infection Cathepsin D is Absent from Cytosolic M. Tuberculosis

Immuno-gold labelling of cathepsin D on DC infected with M. tuberculosis for 96 hours. Inside lysosomes cathepsin D is present but cytosolic bacteria are not labelled (for comparison see FIG. 1C).

### (B) M. Leprae Translocates to the Cytosol

Electron micrograph of a DC infected with M. leprae for 7 days labelled with LAMP-1 and showing a cytosolic location. Note the membranous structures present in the mitochondria showing the capacity of the cryo-sectioning technique to demonstrate membranes, which are absent around M. leprae bacteria.

### (C) Tomogram Demonstrates Phagosomal Membrane Early Infection

A 60 nm cryosection of a DC infected with M. tuberculosis for 48 hours, immuno-labelled for LAMP-1 with 10 nm gold particles. 1 reconstruction was made using weighted back projection using the IMOD software from a −60 degree to +60 degree tilt series taken in 1 degree increments. Note the membranous structures of the Golgi stack and the phagosomal membrane. A 5 nm thick tomographic slice taken from this series is available in FIG. 4A.

### (D) Tomogram of a Cytosolic M. Tuberculosis

A 200 nm thick cryosection of DCs infected with M. tuberculosis for 96 hours immuno-labelled for LAMP-1 with 10 nm gold particles. The reconstruction was made from a −60 degree to +60 degree tilt series taken in 1 degree increments. The reconstruction was made using weighted back projection using the IMOD software. The specimens were sectioned in thick (200 nm) sections to enlarge the chance of including membranous structures however; no membranes surrounding the bacteria were detected. A 5 nm thick section taken from this series is available in FIG. 4C

L: lysosomes, M: mitochondrium, asterisk: mycobacteria in phagolysosomes, encircled asterisks: cytosolic mycobacteria. All images are from cryo-immunogold labelled cryo-sections. Bar: C) 100 nm and D) 200 nm.

### Supplementary FIG. 3.

Also in Macrophages M. Tuberculosis Translocate from the Phagosome to the Cytosol.

### (A) No Transferrin Receptor Present on M. Tuberculosis Phagosomes

Macrophages infected for 48 hours with M. tuberculosis labelled for transferrin receptor. Transferrin receptor is present on small vesicles but absent from the phagolysosomal membrane.

(B) Translocation of M. Tuberculosis into the Cytosol in Macrophages

Macrophage infected for 96 hours with M. tuberculosis labelled for LAMP-1. The lysosome (L) is labelled for LAMP-1 but the mycobacteria reside in the cytosol. Asterisk: mycobacteria in phagolysosomes, encircled asterisks: cytosolic mycobacteria, M: mitochondrium, L: lysosomes, PM: plasma membrane, bars: A) 300 nm B) 200 nm

### Supplementary FIG. 4.

### FtsZ Protein Demonstrates Replication of Cytosolic M. Tuberculosis

### (A) Cytosolic M. Tuberculosis Replicates in the Cytosol

DCs infected with M. tuberculosis or M. tuberculosis Tn::CFP-10 for 3 and 7 days were immunogold labelled for FtsZ. The amount of gold particles relative to the surface size of bacteria in phagolysosomes, bacteria in cytosol, mitochondria (mito), lysosomes (lyso) and nucleus was determined and the relative labelling index (RLI) calculated according to Mayhew (Mayhew et al., 2002). The bacteria in the cytosol contain increased amounts of FtsZ, demonstrating increased replication of M. tuberculosis in the cytosol, while the CFP-10 mutant of M. tuberculosis replicates in the phagosome.

### (B) Immunogold Labbeling of FtsZ on Forming Septa of M. Tuberculosis

Representative electron micrograph of a phagosomal cluster of dividing M. tuberculosis Tn::CFP-10 in DCs infected for 7 days and immunogold labelled for FtsZ (red arrow heads). The FtsZ is specifically labelled at the forming septum between 2 longitudinal sectioned bacteria and in cross sections of some bacteria in the bacterial cytosol, possibly close to a septum. Asterisk: mycobacteria in phagolysosome, bar: 300 nm

### Supplementary FIG. 5

Epon Embedded DCs Infected with M. Tuberculosis

### (A) No Apoptotic Cells Present in Overview of 96 h Infection in DCs.

Low magnification of an ultra-thin section of epon embedded DCs infected with M. tuberculosis for 96 hours. In various cells bacteria can be seen from which the boxed area is enlarged in (B).

### (B) Cellular Membranes Visualized in Epon Embedded DCs.

Higher magnification of boxed area in A) showing cluster M. tuberculosis in a DC 96 hours after infection. Although the membranes of the endoplasmatic reticulum and various lysosomes can be recognized, no host membrane around the bacteria is detectable.

### C) Epon Embedded Cytosolic and Phagosomal M. Tuberculosis

High magnification of M. tuberculosis in a DC 96 hours after infection. The lower cluster consisting of 2 bacteria appears to be in a membrane enclosed compartment possible surrounded by the electron lucent space. The single bacterium appears to be cytosolic since clear membrane structures and the electron lucent space are absent. Epon embedding, contrasting and sectioning was performed as done routinely (McDonough et al., 1993) M: mitochondria, PM: plasma membrane, ER endoplasmic reticulum, bars: A) 3 um; B) 400 nm and C) 300 nm.

### REFERENCE LIST

Ackerman, A. L., Kyritsis, C., Tampe, R., and Cresswell, P. (2003). Early phagosomes in dendritic cells form a cellular compartment sufficient for cross presentation of exogenous antigens. Proc Natl Acad Sci USA 100, 12889-12894.
Adams, L. B., Scollard, D. M., Ray, N. A., Cooper, A. M., Frank, A. A., Orme, I. M., and Krahenbuhl, J. L. (2002). The study of Mycobacterium leprae infection in interferon-gamma gene—disrupted mice as a model to explore the immunopathologic spectrum of leprosy. J Infect Dis 185 Suppl 1, S1-S8.
Armstrong, J. A. and Hart, P. D. (1971). Response of cultured macrophages to Mycobacterium tuberculosis, with observations on fusion of lysosomes with phagosomes. J Exp Med 134, 713-740.
Behr, M. A., Wilson, M. A., Gill, W. P., Salamon, H., Schoolnik, G. K., Rane, S., and Small, P. M. (1999). Comparative genomics of BCG vaccines by whole-genome DNA microarray. Science 284, 1520-1523.
Berthet, F. X., Rasmussen, P. B., Rosenkrands, I., Andersen, P., and Gicquel, B. (1998). A Mycobacterium tuberculosis operon encoding ESAT-6 and a novel low-molecular-mass culture filtrate protein (CFP-10). Microbiology 144 (Pt 11), 3195-3203.
Bouley, D. M., Ghori, N., Mercer, K. L., Falkow, S., and Ramakrishnan, L. (2001). Dynamic nature of host-pathogen interactions in Mycobacterium marinum granulomas. Infect Immun 69, 7820-7831.
Brodin, P., Majlessi, L., Marsollier, L., de Jonge, M. I., Bottai, D., Demangel, C., Hinds, J., Neyrolles, O., Butcher, P. D., Leclerc, C., Cole, S. T., and Brosch, R. (2006). Dissection of ESAT-6 system 1 of Mycobacterium tuberculosis and impact on immunogenicity and virulence. Infect Immun 74, 88-98.
Brosch, R., Gordon, S. V., Marmiesse, M., Brodin, P., Buchrieser, C., Eiglmeier, K., Garnier, T., Gutierrez, C., Hewinson, G., Kremer, K., Parsons, L. M., Pym, A. S., Samper, S., van Soolingen, D., and Cole, S. T. (2002). A new evolutionary scenario for the Mycobacterium tuberculosis complex. Proc Natl Acad Sci USA 99, 3684-3689.
Clemens D. L., Horwitz M. A. (1995). Characterization of the Mycobacterium tuberculosis phagosome and evidence that phagosomal maturation is inhibited. J Exp Med. 181(1):257-70.
Decatur, A. L. and Portnoy, D. A. (2000). A PEST-like sequence in listeriolysin O essential for Listeria monocytogenes pathogenicity. Science 290, 992-995.
Derrick, S. C., and Morris, S. L. (2007). The ESAT6 protein of Mycobacterium tuberculosis induces apoptosis of macrophages by activating caspase expression. Cell Microbiol 9, 1547-1555.
Flynn, J. L. and Chan, J. (2001). Immunology of tuberculosis. Annu Rev Immunol 19, 93-129.
Fortune, S. M., Jaeger, A., Sarracino, D. A., Chase, M. R., Sassetti, C. M., Sherman, D. R., Bloom, B. R., and Rubin, E. J. (2005). Mutually dependent secretion of proteins required for mycobacterial virulence. Proc Natl Acad Sci USA 102, 10676-10681.
Gao, L. Y., Pak, M., Kish, R., Kajihara, K., and Brown, E. J. (2006). A mycobacterial operon essential for virulence in vivo and invasion and intracellular persistence in macrophages. Infect Immun 74, 1757-1767.
Glomski, I. J., Decatur, A. L., and Portnoy, D. A. (2003). Listeria monocytogenes mutants that fail to compartmentalize listerolysin 0 activity are cytotoxic, avirulent, and unable to evade host extracellular defenses. Infect Immun 71, 6754-6765.
Glomski, I. J., Gedde, M. M., Tsang, A. W., Swanson, J. A., and Portnoy, D. A. (2002). The Listeria monocytogenes hemolysin has an acidic pH optimum to compartmentalize activity and prevent damage to infected host cells. J Cell Biol 156, 1029-1038.
Gordon, S. V., Brosch, R., Billault, A., Garnier, T., Eiglmeier, K., and Cole, S. T. (1999). Identification of variable regions in the genomes of tubercle bacilli using bacterial artificial chromosome arrays. Mol Microbiol 32, 643-655.
Grode, L., Seiler, P., Baumann, S., Hess, J., Brinkmann, V., Nasser Eddine, A., Mann, P., Goosmann, C., Bandermann, S., Smith, D., Bancroft, G. J., Reyrat, J. M., van Soolingen, D., Raupach, B., and Kaufmann, S. H. (2005). Increased vaccine efficacy against tuberculosis of recombinant Mycobacterium bovis bacille Calmette-Guerin mutants that secrete listeriolysin. J Clin Invest 115, 2472-2479.
Guermonprez, P., Saveanu, L., Kleijmeer, M., Davoust, J., Van Endert, P., and Amigorena, S. (2003). ER-phagosome fusion defines an MHC class I cross-presentation compartment in dendritic cells. Nature 425, 397-402.
Guinn, K. M., Hickey, M. J., Mathur, S. K., Zakel, K. L., Grotzke, J. E., Lewinsohn, D. M., Smith, S., and Sherman, D. R. (2004a). Individual RD1-region genes are required for export of ESAT-6/CFP-10 and for virulence of Mycobacterium tuberculosis. Mol Microbiol 51, 359-370.
Guinn, K. M., Hickey, M. J., Mathur, S. K., Zakel, K. L., Grotzke, J. E., Lewinsohn, D. M., Smith, S., and Sherman, D. R. (2004b). Individual RD1-region genes are required for export of ESAT-6/CFP-10 and for virulence of Mycobacterium tuberculosis. Mol Microbiol 51, 359-370.
Harboe, M., Oettinger, T., Wiker, H. G., Rosenkrands, I., and Andersen, P. (1996. Evidence for occurrence of the ESAT-6 protein in Mycobacterium tuberculosis and virulent Mycobacterium bovis and for its absence in Mycobacterium bovis BCG. Infect Immun 64, 16-22.
Houde, M., Bertholet, S., Gagnon, E., Brunet, S., Goyette, G., Laplante, A., Princiotta, M. F., Thibault, P., Sacks, D., and Desjardins, M. (2003). Phagosomes are competent organelles for antigen cross-presentation. Nature 425, 402-406.
Hsu, T., Hingley-Wilson, S. M., Chen, B., Chen, M., Dai, A. Z., Morin, P. M., Marks, C. B., Padiyar, J., Goulding, C., Gingery, M., Eisenberg, D., Russell, R. G., Derrick, S. C., Collins, F. M., Morris, S. L., King, C. H., and Jacobs, W. R., Jr. (2003). The primary mechanism of attenuation of bacillus Calmette-Guerin is a loss of secreted lytic function required for invasion of lung interstitial tissue. Proc Natl Acad Sci USA 100, 12420-12425.
Kang, P. B., Azad, A. K., Torrelles, J. B., Kaufman, T. M., Beharka, A., Tibesar, E., DesJardin, L. E., and Schlesinger, L. S. (2005). The human macrophage mannose receptor directs Mycobacterium tuberculosis lipoarabinomannan-mediated phagosome biogenesis. J Exp Med 202, 987-999.
Keane, J., Balcewicz-Sablinska, M. K., Remold, H. G., Chupp, G. L., Meek, B. B., Fenton, M. J., and Kornfeld, H. (1997). Infection by Mycobacterium tuberculosis promotes human alveolar macrophage apoptosis. Infect. Immun. 65, 298-304.
Kerr, J. F., Wyllie, A. H., and Currie, A. R. (1972). Apoptosis: a basic biological phenomenon with wide-ranging implications in tissue kinetics. Br. J. Cancer 26, 239-257.
Kremer, J. R., Mastronarde, D. N., and McIntosh, J. R. (1996). Computer visualization of three-dimensional image data using IMOD. J. Struct. Biol. 116, 71-76.
Lazarevic, V., Nolt, D., and Flynn, J. L. (2005). Long-term control of Mycobacterium tuberculosis infection is mediated by dynamic immune responses. J Immunol 175, 1107-1117.
Leake, E. S., Myrvik, Q. N., and Wright, M. J. (1984). Phagosomal membranes of Mycobacterium bovis BCG-immune alveolar macrophages are resistant to disruption by Mycobacterium tuberculosis H37Rv. Infect Immun 45, 443-446.
Lee, J., Remold, H. G., Ieong, M. H., and Kornfeld, H. (2006). Macrophage apoptosis in response to high intracellular burden of Mycobacterium tuberculosis is mediated by a novel caspase-independent pathway. J. Immunol. 176, 4267-4274.
Lewis, K. N., Liao, R., Guinn, K. M., Hickey, M. J., Smith, S., Behr, M. A., and Sherman, D. R. (2003). Deletion of RD1 from Mycobacterium tuberculosis mimics bacille Calmette-Guerin attenuation. J Infect Dis 187, 117-123.
Majlessi, L., Brodin, P., Brosch, R., Rojas, M. J., Khun, H., Huerre, M., Cole, S. T., and Leclerc, C. (2005). Influence of ESAT-6 secretion system 1 (RD1) of Mycobacterium tuberculosis on the interaction between mycobacteria and the host immune system. J. Immunol. 174, 3570-3579.
Margolin, W. (2005). FtsZ and the division of prokaryotic cells and organelles. Nat. Rev. Mol. Cell. Biol. 6, 862-871.
McDonough, K. A., Kress, Y., and Bloom, B. R. (1993). Pathogenesis of tuberculosis: interaction of Mycobacterium tuberculosis with macrophages. Infect. Immun. 61, 2763-2773.
Mostowy, S., Cousins, D., Brinkman, J., Aranaz, A., and Behr, M. A. (2002). Genomic deletions suggest a phylogeny for the Mycobacterium tuberculosis complex. J Infect Dis 186, 74-80.
Myrvik, Q. N., Leake, E. S., and Wright, M. J. (1984). Disruption of phagosomal membranes of normal alveolar macrophages by the H37Rv strain of Mycobacterium tuberculosis. A correlate of virulence. Am Rev Respir Dis 129, 322-328.
Orme, I. (2004). Adaptive immunity to mycobacteria. Curr Opin Microbiol 7, 58-61.
Peters, P. J., Bos, E. and Griekspoor, A. (2006) Cryo-Immunogold Electron Microscopy; including 11 videos on line. Current Protocols in Cell Biology 4.7.1-4.7.19 by John Wiley & Sons, Inc.
Peters, P. J., Neefjes, J. J., Oorschot, V., Ploegh, H. L., and Geuze, H. J. (1991). Segregation of MHC class II molecules from MHC class I molecules in the Golgi complex for transport to lysosomal compartments. Nature 349, 669-676.
Philipp, W. J., Nair, S., Guglielmi, G., Lagranderie, M., Gicquel, B., and Cole, S. T. (1996). Physical mapping of Mycobacterium bovis BCG pasteur reveals differences from the genome map of Mycobacterium tuberculosis H37Rv and from M. bovis. Microbiology 142 (Pt 11), 3135-3145.
Pizarro-Cerda, J. and Cossart, P. (2006). Bacterial adhesion and entry into host cells. Cell 124, 715-727.
Placido, R., Mancino, G., Amendola, A., Mariani, F., Vendetti, S., Piacentini, M., Sanduzzi, A., Bocchino, M. L., Zembala, M., and Colizzi, V. (1997). Apoptosis of human monocytes/macrophages in Mycobacterium tuberculosis infection. J. Pathol. 181, 31-38.
Porcelli, S., Morita, C. T., and Brenner, M. B. (1992). CD1b restricts the response of human CD4-8-T lymphocytes to a microbial antigen. Nature 360, 593-597.
Ramakrishnan, L., Federspiel, N. A., and Falkow, S. (2000). Granuloma-specific expression of Mycobacterium virulence proteins from the glycine-rich PE-PGRS family. Science 288, 1436-1439.
Riendeau, C. J., and Kornfeld, H. (2003). THP-1 cell apoptosis in response to Mycobacterial infection. Infect. Immun. 71, 254-259.
Roy, C. R. and Tilney, L. G. (2002). The road less traveled: transport of Legionella to the endoplasmic reticulum. J Cell Biol 158, 415-419.
Russell, D. G. (2001). Mycobacterium tuberculosis: here today, and here tomorrow. Nat Rev Mol Cell Biol 2, 569-577.
Russell, D. G., Mwandumba, H. C., and Rhoades, E. E. (2002). Mycobacterium and the coat of many lipids. J Cell Biol 158, 421-426.
Schnupf, P., Portnoy, D. A., and Decatur, A. L. (2006). Phosphorylation, ubiquitination and degradation of listeriolysin O in mammalian cells: role of the PEST-like sequence. Cell Microbiol 8, 353-364.
Schuerch, D. W., Wilson-Kubalek, E. M., and Tweten, R. K. (2005). Molecular basis of listeriolysin 0 pH dependence. Proc. Natl. Acad. Sci. USA 102, 12537-12542.
Stamm, L. M., Morisaki, J. H., Gao, L. Y., Jeng, R. L., McDonald, K. L., Roth, R., Takeshita, S., Heuser, J., Welch, M. D., and Brown, E. J. (2003). Mycobacterium marinum escapes from phagosomes and is propelled by actin-based motility. J Exp Med 198, 1361-1368.
Stamm, L. M., Pak, M. A., Morisaki, J. H., Snapper, S. B., Rottner, K., Lommel, S., and Brown, E. J. (2005). Role of the WASP family proteins for Mycobacterium marinum actin tail formation. Proc Natl Acad Sci USA 102, 14837-14842.
Stanley, S. A., Raghavan, S., Hwang, W. W., and Cox, J. S. (2003). Acute infection and macrophage subversion by Mycobacterium tuberculosis require a specialized secretion system. Proc Natl Acad Sci USA 100, 13001-13006.
Stevens, J. M., Galyov, E. E., and Stevens, M. P. (2006). Actin-dependent movement of bacterial pathogens. Nat Rev Microbiol 4, 91-101.
Tekaia, F., Gordon, S. V., Garnier, T., Brosch, R., Barrell, B. G., and Cole, S. T. (1999). Analysis of the proteome of Mycobacterium tuberculosis in silico. Tuber. Lung Dis 79, 329-342.
Touret, N., Paroutis, P., Terebiznik, M., Harrison, R. E., Trombetta, S., Pypaert, M., Chow, A., Jiang, A., Shaw, J., Yip, C., Moore, H. P., van der Wel, N., Houben, D., Peters, P. J., de Chastellier, C., Mellman, I., and Grinstein, S. (2005). Quantitative and dynamic assessment of the contribution of the ER to phagosome formation. Cell 123, 157-170.
van der Wel, N. N., Sugita, M., Fluitsma, D. M., Cao, X., Schreibelt, G., Brenner, M. B., and Peters, P. J. (2003). CD1 and major histocompatibility complex II molecules follow a different course during dendritic cell maturation. Mol Biol Cell 14, 3378-3388.
Vergne, I., Chua, J., Singh, S. B., and Deretic, V. (2004). Cell biology of mycobacterium tuberculosis phagosome. Annu Rev Cell Dev Biol 20, 367-394.
Waterman, S. R. and Holden, D. W. (2003). Functions and effectors of the Salmonella pathogenicity island 2 type III secretion system. Cell Microbiol 5, 501-511.
Zamboni, D. S., Kobayshi, K. S., Kohlsdorf, T., Ogura, Y., Long, E. M., Vance, R. E., Kuida, K., Mariathasan, S., Dixit, V. M., Flavell, R. A., Dietch, W. F., and Roy, C. R. (2006). The Bircle cytosolic pattern-recognition receptor contributes to the detection and control of Legionella pneumophila infection. Nat Immunol 7, 318-325.

### Table 1 of Example 1

Immuno gold labelling of several marker specific for different cellular compartments which were present (+) or absent (−) on membrane of M. tuberculosis or M. leprae the phagosomal in DCs infected for 2 hours.

| Compartment | marker | M. tuberculosis | M. leprae |
|---|---|---|---|
| ER | PDI | − | − |
| | MHC I | − | − |
| | TAP | − | − |
| Early endosome | TfR | − | − |
| | EEA1 | − | − |
| Late endosome | M6PR | − | − |
| Lysosome | CD63 | + | + |
| | LAMP-1 | + | + |
| | LAMP-2 | + | + |

### Table 1 of Example 2

Immunogold labelling of several markers specific for different cellular compartments which were present (+) or absent (−) on M. tuberculosis or M. leprae containing phagosomes in DCs infected for 2 hours.

| Compartment | Marker | M. tuberculosis | M. leprae |
|---|---|---|---|
| ER | PDI | − | − |
| | MHC I | − | − |
| | TAP | − | − |
| early endosome | TfR | − | − |
| | EEA1 | − | − |
| late endosome | M6PR | − | − |
| lysosome | CD63 | + | + |
| | LAMP-1 | + | + |
| | LAMP-2 | + | + |
| | Cathepsin D | + | + |

1. A method for determining whether a product of a gene of a mycobacterium is involved in translocation of said mycobacterium from the phagosome to the cytosol of a host cell, said method comprising altering said gene product and/or expression of said gene product in said mycobacterium and determining whether said translocation of said mycobacterium in said host cell is affected.

2. A method according to claim 1, wherein said gene is a gene from a region of difference (RD) between mycobacterium tuberculosis and Bacille Calmette Guerin (BCG), or from a corresponding region in another mycobacterium species.

3. A method according to claim 2, wherein said other mycobacterium species is selected from mycobacterium bovis, leprae, smegmatis or marinum.

4. A method according to any one of claims 1-3, wherein said gene is derived from RDI, preferably CFPIO, ESAT6 or EspA.

5. A method for reducing the phago-cytosolic translocation of a mycobacterium comprising at least reducing the expression of CFPIO, ESAT6 or EspA in said mycobacterium.

6. A method for enhancing phago-cytosolic translocation of a CFPIO, ESAT6 and/or EspA deficient mycobacterium, said method comprising providing said mycobacterium with CFPIO, ESAT6 and/or EspA.

7. A method for generating a recombinant BCG strain comprising providing BCG or a derivative thereof with CFPIO, ESAT6 and/or EspA.

8. A method for producing a mycobacterium that is substantially deficient in phago-cytosolic translocation comprising functionally reducing the expression of CFPIO, ESAT6 and/or EspA in said mycobacterium.

9. A method according to claim 8, wherein the gene encoding CFPIO, ESAT6 and/or EspA is mutated and/or removed such that substantially no functional CFPIO, ESAT6 and/or EspA is produced by said mycobacterium.

10-11. (canceled)

12. An attenuated mycobacterium comprising a nucleic acid encoding CFPIO, ESAT6 and/or EspA further comprising a heterologous nucleic acid for inhibiting cytosolic replication and/or cytosolic translocation of said mycobacterium.

13. An attenuated mycobacterium according to claim 12, wherein said heterologous nucleic acid comprises a regulatable promoter.

14-18. (canceled)

19. A mycobacterium according to claims 12 or 13, wherein said viral protein is a human virus protein or an animal virus protein.

20. (canceled)

21. A killed or attenuated mycobacterium according to claim 12.

22. An immunogenic composition produced from a mycobacterium according to claims 12 or 13.

23-25. (canceled)

26. An immunogenic composition comprising a mycobacterium according to claims 12 or 13.

27. (canceled)

28. A method for providing a mycobacterium with the capacity to translocate from a phagosome to the cytosol of a host cell, or to enhance said capacity, comprising infecting the mycobacterium with a nucleic acid encoding CFPIO, ESAT6 and/or EspA.

29-32. (canceled)

33. A method for selecting a mycobacterium for the preparation of a vaccine comprising infecting cells permissive for said mycobacterium in vitro with said collection and selecting from said collection a mycobacterium which translocates to the cytosol of infected cells.

34. A method for obtaining an immune response in an individual comprising providing said individual with a mycobacterium according to claims 12 or 13.

35-43. (canceled)
